(19) United States
(12) Patent Application Publication
(10) Pub. No.: US 2025/0120739 A1
(43) Pub. Date: Apr. 17, 2025

(54) ATRIAL SEPTUM PERFORATION DEVICE AND BODY TISSUE PERFORATION DEVICE

(71) Applicant: NIPRO CORPORATION, Settsu-shi, Osaka (JP)

(72) Inventor: Koji KAMEOKA, Settsu-shi, Osaka (JP)

(21) Appl. No.: 18/568,494

(22) PCT Filed: Jun. 9, 2022
(86) PCT No.: PCT/JP2022/023255

§ 371 Date: Dec. 8, 2023

(30) Foreign Application Priority Data
Jun. 9, 2021 (JP) 2021-096890
Jul. 16, 2021 (JP) 2021-117665

(51) Int. Cl. A61B 17/34; A61B 17/00; A61B 90/00

(52) U.S. Cl. CPC A61B 17/3401; A61B 17/00234; A61B 17/3417; A61B 90/08; A61B 2017/00247; A61B 2017/00292; A61B 2017/00929; A61B 2090/0811

(57) ABSTRACT

An atrial septum perforation device including: at a distal end of a shaft, a perforation head part that performs perforation operation on a body tissue; and, at a proximal end of the shaft, an operation handle gripped by a practitioner. A distal end portion of the shaft is a shaft curved part that is curved in one direction. The operation handle includes a rotational operation assisting surface that extends vertically or horizontally when a direction of curving of the shaft curved part is set to a roughly 4 o'clock direction around a body axis parallel line of a patient so that the perforation head part is turned to a fossa ovalis.

### TECHNICAL FIELD

This invention relates to an atrial septum perforation device used for forming a patent foramen in the atrial septum, a body tissue perforation device used for forming a patent foramen in body tissue, and a perforation device assembly in which a dilator and a sheath are combined with the body tissue perforation device.

### BACKGROUND ART

Conventionally, one of the treatment methods for atrial fibrillation is to cauterize an appropriate site in the left atrium with an ablation catheter. The ablation catheter may be inserted from the right atrium into the left atrium by passing through the atrial septum, in which case a patent foramen passing through the atrial septum must be formed in advance. For perforation of the atrial septum, for example, the Brockenbrough method, in which the fossa ovalis is punctured, is a well-known method of forming the patent foramen. In the Brockenbrough method, a perforation device such as the medical instrument shown in Japanese Patent No. JP-B-6416084 (Patent Document 1), which has a structure in which a perforation head part for performing perforation operation is provided at the distal end of the shaft, is used. The perforation head part at the distal end of the shaft inserted into the right atrium perforates the fossa ovalis, thereby forming a patent foramen.

### BACKGROUND ART DOCUMENT

### Patent Document

Patent Document 1: JP-B-6416084

### SUMMARY OF THE INVENTION

### Problem the Invention Attempts to Solve

Meanwhile, in the procedure of forming a patent foramen in body tissue in the body using a perforation device, it is not possible to directly visually check the state of the distal end portion of the perforation device that is percutaneously inserted into the body. Therefore, the practitioner performs the procedure while imagining the state of the distal end portion of the perforation device based on, for example, the orientation and position of an operation handle provided to the proximal end portion of the perforation device, the orientation (the shape) of the hand holding the operation handle, and the orientation and position of the perforation device with respect to the dilator and the sheath, or the like.

However, with the conventional perforation device, it is difficult to estimate the state of the distal end portion, which has been inserted into the body, at the proximal end portion located outside the body, and there is a possibility that the procedure becomes difficult. Although an X-ray monitor or the like is used, at least at present, the actual situation is that the X-ray image is only for reference.

Also, in the perforation device of Patent Document 1, an opening is formed on the side surface of the distal portion of the tube, and a contrast medium, physiological saline, etc. can be released through the opening from the lumen of the tube, for example. Besides, in the perforation of the fossa ovalis, in addition to fluoroscopy using contrast imaging and feedback from the perforation device, pressure measurement is employed as a means of grasping the insertion of the tube into the left atrium after perforation. That is, by measuring the pressure in the lumen of the tube that is connected to the atrium through the opening, the distal end position of the perforation device is grasped based on changes in pressure. Furthermore, there is a request to grasp as accurately as possible the state of perforation, such as whether the distal end of the perforation device is pressed against the fossa ovalis or whether the distal end of the perforation device perforates and passes through the fossa ovalis.

However, the perforation device of Patent Document 1 has only a lateral opening. Thus, it is difficult to accurately grasp whether the distal end is pressed against the fossa ovalis because, for example, simply pressing the distal end against the fossa ovalis does not cause a pressure change in the lumen.

It is therefore one object of the present invention according to some preferred embodiments to provide an atrial septum perforation device or the like of novel structure that enables a practitioner to easily grasp the state of the distal end portion inserted into the body.

It is another object of the present invention according to some preferred embodiments to provide a body tissue perforation device of novel structure that enables the state of perforation to be grasped more accurately when perforating a body tissue.

### Means for Solving the Problem

Hereinafter, preferred embodiments for grasping the present invention will be described. However, each preferred embodiment described below is exemplary and can be appropriately combined with each other. Besides, a plurality of elements described in each preferred embodiment can be recognized and adopted as independently as possible, or can also be appropriately combined with any element described in other preferred embodiments. By so doing, in the present invention, various other preferred embodiments can be realized without being limited to those described below.

In the procedure to form a patent foramen in the atrial septum perforation using a perforation device, the practitioner inserts a dilator and a sheath along a guide wire from the inferior vena cava to the superior vena cava, then removes the guide wire and inserts the perforation device into the dilator to constitute the perforation device assembly. Next, the practitioner moves the perforation device assembly to the proximal side with the distal end of the perforation device (the perforation head part) housed in the dilator, brings the distal end of the dilator into contact with the fossa ovalis of the atrial septum, and directs the perforation head part to the fossa ovalis. At this point, before moving the perforation device assembly to the proximal side, it is necessary to rotate the perforation device assembly so that the distal end portion of the curved perforation device assembly is turned to the direction that is rotated about-30 degrees with respect to the horizontal when viewed in the axial direction from the proximal side to the distal side (the so-called 4 o'clock direction). The practitioner then pushes the perforation device to the distal side with respect to the dilator, and causes the perforation head part to protrude from the distal end of the dilator and to perform perforation operation on the fossa ovalis in a state of contact, thereby forming a patent foramen in the fossa ovalis.

However, with conventional atrial septum perforation devices, it is difficult for the practitioner to grasp the state of the distal end portion being directed to the approximately 4 o'clock direction, which may cause difficulty in the procedure, such as failure to bring the distal end portion into contact with the fossa ovalis or failure to maintain the distal end portion in contact with the fossa ovalis.

Therefore, a first preferred embodiment provides an atrial septum perforation device comprising: a shaft; a perforation head part that is provided to a distal end of the shaft and is configured to perform perforation operation on a body tissue; and an operation handle that is provided to a proximal end of the shaft and is configured to be gripped by a practitioner, wherein a distal end portion of the shaft comprises a shaft curved part that is curved in one direction, the operation handle includes a rotational operation assisting surface that extends vertically or horizontally when a direction of curving of the shaft curved part is set to a substantially 4 o'clock direction around a body axis parallel line of a patient such that the perforation head part is turned to a fossa ovalis.

According to the atrial septum perforation device structured following the present preferred embodiment, by holding the rotational operation assisting surface of the operation handle vertically or horizontally, it is possible to keep the direction of curving of the shaft curved part in the approximately 4 o'clock direction around the body axis parallel line so that the perforation head part is directed to the fossa ovalis. Since it is easy for the practitioner to grasp whether the rotational operation assisting surface is vertical or horizontal, the orientation of the perforation head part (the direction of curving of the shaft) in the body can be easily grasped by the rotational operation assisting surface provided on the operation handle outside the body. It is also easy to continuously keep the orientation of the perforation head part positioned with respect to the fossa ovalis, thereby preventing the orientation of the perforation head part from changing during the procedure.

A second preferred embodiment provides the atrial septum perforation device according to the first preferred embodiment, wherein the operation handle has a plate shape, and the rotational operation assisting surface is provided on an outer circumferential surface of the operation handle and is inclined relative to a plate thickness direction of the operation handle.

According to the atrial septum perforation device structured following the present preferred embodiment, the operation handle has a plate shape, thereby enabling operations such as rotating the shaft to be performed easily and accurately. In addition, the rotational operation assisting surface is provided as a relatively inclined surface that is not perpendicular or parallel to the plate thickness direction. This makes it easy to grasp the rotational operation assisting surface by tactile feeling while gripping the operation handle.

A third preferred embodiment provides the atrial septum perforation device according to the second preferred embodiment, wherein on the outer circumferential surface of the operation handle, the rotational operation assisting surface is partially provided in the plate thickness direction of the operation handle, and on the outer circumferential surface of the operation handle, a gripping surface is provided, the gripping surface being adjacent to the rotational operation assisting surface in the plate thickness direction of the operation handle while extending substantially parallel to the plate thickness direction of the operation handle.

According to the atrial septum perforation device structured following the present preferred embodiment, before turning the distal end portion of the perforation device toward the approximately 4 o'clock direction, the gripping surface receives the force to grip the operation handle, while when the distal end portion of the device is turned toward the approximately 4 o'clock direction, the rotational operation assisting surface receives the force to grip the operation handle. This prevents the operation handle from being accidentally tilted by the force to grip the operation handle, thereby improving stability of the operation handle.

A fourth preferred embodiment provides the atrial septum perforation device according to the second or third preferred embodiment, wherein the plate thickness direction of the operation handle is substantially orthogonal to a plane including a central axis of the shaft curved part.

According to the atrial septum perforation device structured following the present preferred embodiment, the direction of curving of the shaft curved part can be grasped by the plate thickness direction of the operation handle. In particular, in the state before the shaft curved part is turned to the approximately 4 o'clock direction, the direction of curving of the shaft curved part can be easily grasped by the plate thickness direction of the operation handle.

A fifth preferred embodiment provides the atrial septum perforation device according to the fourth preferred embodiment, wherein the rotational operation assisting surface is provided on the outer circumferential surface of the operation handle located on a side opposite to the direction of curving of the shaft curved part when viewed in an axial direction from a proximal side to a distal side.

According to the atrial septum perforation device structured following the present preferred embodiment, when the practitioner grips the operation handle, the practitioner's thumb, which has an acute sense of touch, can touch the rotational operation assisting surface, making it easy for the practitioner to grasp the state of the rotational operation assisting surface extending vertically or horizontally.

A sixth preferred embodiment provides the atrial septum perforation device according to any one of the first to fifth preferred embodiments, wherein the shaft is configured to be inserted through a dilator, and a marker part is provided on a proximal end side of the shaft, the marker part indicating in a visually confirmable manner that a distal end of the perforation head part is housed in the dilator without protruding from the dilator.

According to the atrial septum perforation device structured following the present preferred embodiment, it can be easily grasped that the perforation head part, which is provided at the distal end of the shaft and inserted into the body, is housed without protruding from the dilator by visually checking the marker part, which is provided on the proximal end side of the shaft outside the body.

A seventh preferred embodiment provides the atrial septum perforation device according to any one of the first to sixth preferred embodiments, wherein the shaft is configured to be inserted through a dilator, and the shaft includes a positioning part configured to position the shaft with respect to the dilator in a circumferential direction.

According to the atrial septum perforation device structured following the present preferred embodiment, the shaft of the perforation device includes the positioning part for positioning the shaft with respect to the dilator in the circumferential direction. Thus, when the operation handle is rotated, not only does the shaft rotate, but the dilator also rotates with the shaft. This makes it possible to prevent misorientation of the shaft and the dilator due to their relative rotation at the insertion portion into the body, where direct visual confirmation is not possible.

For example, when pulling the distal end portion of the perforation device from the superior vena cava into the right atrium, it is necessary to house the perforation head part in the dilator so as to prevent the perforation head part from protruding from the distal end of the dilator and damaging the body tissue. Thus, in the past, the perforation head part was housed in the dilator by pulling the perforation device to the proximal side with respect to the dilator by one finger length to prevent the perforation head part from protruding from the dilator to the distal end side.

However, it was difficult to maintain a constant position of the perforation device relative to the dilator because the amount of pulling the perforation device to the proximal side with respect to the dilator was an ambiguous amount, described as “one finger length”. As a result, there may be a risk of making the procedure more difficult, for example, the amount of movement of the perforation device to the proximal side with respect to the dilator was insufficient, causing the perforation head part to protrude slightly from the distal end of the dilator, or the distal end portion of the curved perforation device was moved too far to the proximal side with respect to the dilator, causing the dilator and the sheath, which have been pre-shaped with respective predetermined curved parts or the like, to be deformed into unintended shapes by the perforation device, or the like.

Therefore, an eighth preferred embodiment provides a body tissue perforation device comprising: a shaft; a perforation head part that is provided to a distal end of the shaft and is configured to perform perforation operation on a body tissue; and an operation handle that is provided to a proximal end of the shaft and is configured to be gripped by a practitioner, wherein the shaft is configured to be inserted through a dilator, and a marker part is provided on a proximal end side of the shaft, the marker part indicating in a visually confirmable manner that a distal end of the perforation head part is housed in the dilator without protruding from the dilator.

According to the body tissue perforation device structured following the present preferred embodiment, it can be easily grasped that the perforation head part, which is provided at the distal end of the shaft and inserted into the body, is housed without protruding from the dilator by visually checking the marker part, which is provided on the proximal end side of the shaft located outside the body.

A ninth preferred embodiment provides the body tissue perforation device according to the eighth preferred embodiment, wherein alignment between the marker part and a proximal end of the dilator indicates that the distal end of the perforation head part is housed in the dilator.

According to the body tissue perforation device structured following the present preferred embodiment, the perforation head part can be easily and reliably housed in the dilator by a simple operation of aligning the marker part with the proximal end of the dilator.

A tenth preferred embodiment provides the body tissue perforation device according to the eighth or ninth preferred embodiment, wherein a surface of the shaft is covered by an electrically insulating coating material, and the electrically insulating coating material is partially different in color from other portions such that the marker part is provided on the surface of the shaft.

According to the body tissue perforation device structured following the present preferred embodiment, for example, when the perforation head part is supplied with electric power to perform perforation operation, by covering the surface of the shaft with an electrically insulating coating material, it is possible to avoid troubles such as loss of energy due to electric leakage and electric shock. In particular, since the insulating layer is formed by the coating process, the insulating layer can be made thin, thereby obtaining a shaft with a small diameter and minimizing the influence of the insulating layer on the curving deformation characteristics or the like of the shaft.

Besides, the insulating layer is formed on the surface of the shaft by coating. Thus, for example, a marker part can be formed by applying a primary coating while partially masking the proximal end portion of the shaft and then applying a secondary coating to the masked portion with an insulating material of a different color from that of the primary coating. This makes it possible to form the marker part regardless of the material of the coating material, and the degree of freedom in selecting the coating material is less likely to be restricted by providing a marker part.

An eleventh preferred embodiment provides the body tissue perforation device according to any one of the eighth to tenth preferred embodiments, further comprising an extended part extending out on an outer circumferential surface of the shaft from the operation handle, wherein the extended part is allowed to be inserted in a proximal end part of the dilator, and the extended part constitutes the marker part.

According to the body tissue perforation device structured following the present preferred embodiment, the perforation head part is easily and reliably housed in the dilator, for example, by aligning the distal end of the extended part of the operation handle with the proximal end of the dilator. In addition, the perforation head part can protrude from the distal end of the dilator by inserting the extended part into the proximal end part of the dilator.

A twelfth preferred embodiment provides the body tissue perforation device according to any one of the eighth to eleventh preferred embodiments, wherein the marker part visibly indicates that the distal end of the perforation head part is housed in the dilator without protruding from the dilator before a retraction amount of the distal end of the perforation head part into the dilator exceeds 20 mm.

According to the body tissue perforation device structured following the present preferred embodiment, it is possible to prevent the perforation head part from being retracted to a position that is significantly remote from the distal end of the dilator. With this configuration, for example, when the distal end portions of the shaft and the dilator are curved, the curved portion of the shaft and the curved portion of the dilator are prevented from shifting significantly in the length direction, thereby inhibiting deformation of the dilator due to the curved shape of the shaft.

Meanwhile, for example, when rotating the perforation device assembly to turn the distal end portion of the perforation device assembly toward the fossa ovalis in the perforation procedure or the like to form a patent foramen in the atrial septum as described above, the practitioner attempts to rotate the perforation device assembly, including the dilator and the sheath, by operating the operation handle of the perforation device. However, in the conventional perforation device assembly, the perforation device, the dilator, and the sheath are only inserted in a state in which they can rotate relative to one another. Thus, there is a risk that the perforation device may rotate circumferentially with respect to the dilator, or that the perforation device and the dilator may rotate circumferentially with respect to the sheath. As a result, there is a risk that the dilator and the sheath, which have been pre-shaped with respective predetermined curved parts, may be deformed into unintended shapes by the perforation device, thereby making the procedure difficult.

Therefore, a thirteenth preferred embodiment provides a perforation device assembly comprising: a body tissue perforation device comprising: a shaft; a perforation head part that is provided to a distal end of the shaft and is configured to perform perforation operation on a body tissue; and an operation handle that is provided to a proximal end of the shaft and is configured to be gripped by a practitioner; a dilator through which the shaft of the body tissue perforation device is inserted; a sheath through which the dilator is inserted; a first circumferential positioning mechanism mutually positioning the shaft of the body tissue perforation device and the dilator in a circumferential direction; and a second circumferential positioning mechanism mutually positioning the dilator and the sheath in the circumferential direction.

According to the perforation device assembly structured following the present preferred embodiment, the shaft, the dilator, and the sheath of the perforation device are mutually positioned in the circumferential direction. Thus, when the operation handle of the perforation device is rotated, not only does the perforation device rotate, but also the dilator and the sheath rotate. Therefore, the perforation device, the dilator, and the sheath can be rotated in an integrated manner by the rotation operation of the operation handle, thereby preventing misorientation of the perforation device, the dilator, and the sheath due to their relative rotation at the insertion portion into the body, where direct visual confirmation is not possible.

A fourteenth preferred embodiment provides the perforation device assembly according to the thirteenth embodiment, wherein the shaft of the body tissue perforation device includes a shaft curved part, the dilator includes a dilator curved part that has a curved shape corresponding to that of the shaft curved part and through which the shaft curved part is inserted, and the sheath includes a sheath curved part that has a curved shape corresponding to that of the dilator curved part and through which the dilator curved part is inserted.

According to the perforation device assembly structured following the present preferred embodiment, the shaft, the dilator, and the sheath, which have respective curved shapes corresponding to one another, are mutually positioned in the circumferential direction. This makes it possible to prevent the directions of curving of their respective curved parts from shifting from one another in the circumferential direction.

A fifteenth preferred embodiment provides a body tissue perforation device comprising: a tube; and a perforation head part that is provided to a distal end of the tube and is configured to perform perforation operation on a body tissue, wherein the perforation head part includes a through hole, and a lumen of the tube opens to a distal end of the perforation head part through the through hole without being blocked by the perforation head part, and a side hole opens onto a side circumferential surface at a position that is configured not to be covered by the body tissue during the perforation operation performed by the perforation head part such that the lumen of the tube is kept in a communicating state with an outside.

According to the body tissue perforation device structured following the present preferred embodiment, the pressure, which is exerted in the lumen of the tube through the through hole opening at the distal end of the perforation head part that performs the perforation operation, changes depending on the state of perforation, such as before, during, and after the perforation. Thus, for example, by detecting the pressure in the lumen of the tube, it is possible to grasp the state of perforation.

Besides, the side hole is provided at a position that is not covered by the body tissue during the perforation. Thus, the function of releasing fluid into the body or suctioning fluid from the body through the lumen of the tube can be stably maintained by the side hole.

A sixteenth preferred embodiment provides the body tissue perforation device according to the fifteenth preferred embodiment, wherein the side hole is located on a distal side with respect to the distal end of the tube and opens onto an outer circumferential surface.

According to the body tissue perforation device structured following the present preferred embodiment, the side hole is provided on a more distal side, thereby enabling fluid such as a contrast medium to be released at a position closer to the distal end of the body tissue perforation device.

A seventeenth preferred embodiment provides the body tissue perforation device according to the fifteenth preferred embodiment, wherein the side hole opens onto an outer circumferential surface of the tube.

According to the body tissue perforation device structured following the present preferred embodiment, the side hole can be provided at an appropriate distance from the tip (the distal end) of the body tissue perforation device. Besides, for example, if the perforation head part has a function of an imaging marker, deterioration in visibility of the perforation head part under the X-ray fluoroscopy due to the formation of the side hole in the perforation head part can be prevented.

An eighteenth preferred embodiment provides the body tissue perforation device according to any one of the fifteenth to seventeenth preferred embodiments, wherein a distal end of an opening of the side hole is located within a range of 0.5 to 5.0 mm from the distal end of the perforation head part to a proximal end side.

According to the body tissue perforation device structured following the present preferred embodiment, the distal end of the opening of the side hole is located at least 0.5 mm away from the distal end of the perforation head part to the proximal end side. This facilitates keeping the side hole open without being covered by the body tissue during perforation. Besides, the distance from the distal end of the opening of the side hole to the distal end of the perforation head part is set to 5.0 mm or less. Thus, for example, when the body tissue perforation device is used by being inserted through a dilator or the like, by exposing the side hole at the distal end side with respect to the dilator or the like without excessively protruding the body tissue perforation device from the dilator or the like, more stable opening is likely to be achieved.

A nineteenth preferred embodiment provides the body tissue perforation device according to any one of the fifteenth to eighteenth preferred embodiments, wherein an outer diameter dimension of the perforation head part is set within a range of 0.5 to 1.0 mm.

According to the body tissue perforation device structured following the present preferred embodiment, the outer diameter dimension of the perforation head part is set to 0.5 mm or more. This makes it possible to form the through hole opening at the distal end of the perforation head part with a sufficient size. In addition, the outer diameter dimension of the perforation head part is set to 1.0 mm or less. This avoids an increase in diameter of the body tissue perforation device, thereby improving insertability into the medical devices such as dilators and the body tissues.

A twentieth preferred embodiment provides the body tissue perforation device according to any one of the fifteenth to nineteenth preferred embodiments, wherein a length dimension of the perforation head part is set within a range of 0.25 to 3.0 mm.

According to the body tissue perforation device structured following the present preferred embodiment, the length dimension of the perforation head part is set to 0.25 mm or more (or even 0.3 mm or more), so that a size favorable for perforation can be obtained. Besides, even when the side hole is formed at a position away from the perforation head part to the proximal side, for example, the degree of freedom in setting the position and the size of the side hole can be sufficiently obtained. In addition, by setting the length dimension of the perforation head part to 3.0 mm or less, reduction in energy loss during the perforation operation can be achieved.

A twenty-first preferred embodiment provides the body tissue perforation device according to any one of the fifteenth to twentieth preferred embodiments, wherein the perforation head part includes a peripheral wall portion having a thickness of not less than 0.1 mm and a length of not less than 0.3 mm.

According to the body tissue perforation device structured following the present preferred embodiment, when the perforation head part having the peripheral wall portion functions as an imaging marker, excellent visibility of the perforation head part can be achieved under the X-ray fluoroscopy.

A twenty-second preferred embodiment provides the body tissue perforation device according to any one of the fifteenth to twenty-first preferred embodiments, wherein an insulating layer is provided on a surface of the perforation head part, the insulating layer covering an outer circumferential surface on a proximal end side of the perforation head part.

According to the body tissue perforation device structured following the present preferred embodiment, the energy discharged from the perforation head part is concentrated on the distal end portion of the perforation head part, thereby improving the efficiency of perforating the body tissue.

A twenty-third preferred embodiment provides the body tissue perforation device according to any one of the first to twenty-second preferred embodiments, further comprising: a dilator through which the tube including the perforation head part is inserted, the dilator being combined with the body tissue perforation device; and a projection length limiting mechanism configured to limit a maximum distance from a distal end of the dilator to the distal end of the perforation head part within a range of 3 to 20 mm.

According to the body tissue perforation device structured following the present preferred embodiment, the maximum projection length of the distal end of the perforation head part from the dilator is set to 3 mm or more by the projection length limiting mechanism, so that the side hole can be opened on the distal end side with respect to the dilator. In addition, the maximum projection length of the distal end of the perforation head part from the dilator is set to 20 mm or less by the projection length limiting mechanism, so that troubles such as excessive projection of the perforation head part from the dilator causing the perforation head part to touch the body tissue other than that to be perforated can be prevented.

### Effect of the Invention

According to some of the preferred embodiments of the present invention, the practitioner can easily grasp the state of the distal end portion of the perforation device or the like inserted into the body.

According to some of the preferred embodiments of the present invention, it is possible to accurately grasp the state of perforation when perforating a body tissue.

### BRIEF DESCRIPTION OF THE DRAWINGS

FIG. 1 is a top plan view showing a high-frequency needle as a first practical embodiment of the present invention according to the first to fourteenth preferred embodiments.

FIG. 2 is a top plan view of an operation handle constituting the high-frequency needle shown in FIG. 1.

FIG. 3 is a bottom plan view of the operation handle shown in FIG. 2.

FIG. 4 is a front view of the operation handle shown in FIG. 2.

FIG. 5 is a rear view of the operation handle shown in FIG. 2.

FIG. 6 is a right side view of the operation handle shown in FIG. 2.

FIG. 7 is a cross sectional view taken along line 7-7 of FIG. 6.

FIG. 8 is a rear view showing the high-frequency needle shown in FIG. 1, with a perforation head part turned to the 4 o'clock direction.

FIG. 9 is a view suitable for explaining a specific example of a treatment device when the high-frequency needle shown in FIG. 1 is used in the Brockenbrough method.

FIG. 10 is a top plan view of a perforation device assembly including the high-frequency needle shown in FIG. 1.

FIG. 11 is a cross sectional view taken along line 11-11 of FIG. 10.

FIG. 12 is a cross sectional view taken along line 12-12 of FIG. 10.

FIG. 13 is a view showing the perforation device assembly shown in FIG. 10 inserted along a guidewire to a superior vena cava.

FIG. 14 is a top plan view of the perforation device assembly shown in FIG. 10, showing an example of how the operation handle is held with the perforation head part directed to the 3 o'clock direction.

FIG. 15 is a front view of the perforation device assembly shown in FIG. 10, showing an example of how the operation handle is held with the perforation head part directed to the 3 o'clock direction.

FIG. 16 is a top plan view of the perforation device assembly shown in FIG. 10, showing an example of how the operation handle is held with the perforation head part directed to the 4 o'clock direction.

FIG. 17 is a front view of the perforation device assembly shown in FIG. 10, showing an example of how the operation handle is held with the perforation head part directed to the 4 o'clock direction.

FIG. 18 is a view showing the perforation device assembly shown in FIG. 10 with its distal end portion inserted into a right atrium and turned to a fossa ovalis.

FIG. 19 is a bottom plan view showing a proximal end portion of a high-frequency needle as a second practical embodiment of the present invention according to the first to fourteenth preferred embodiments.

FIG. 20 is a rear view of an operation handle constituting the high-frequency needle shown in FIG. 19.

FIG. 21 is a bottom plan view showing a proximal end portion of a high-frequency needle as a third practical embodiment of the present invention according to the first to fourteenth preferred embodiments.

FIG. 22 is a right side view of the proximal end portion of the high-frequency needle shown in FIG. 21.

FIG. 23 is a top plan view showing a perforation device assembly as another practical embodiment (a fourth practical embodiment) of the present invention according to the first to fourteenth preferred embodiments.

FIG. 24 is a vertical cross sectional view of a distal end portion of a high-frequency needle as a fifth practical embodiment which is equivalent to the first practical embodiment of the present invention according to the fifteenth to twenty-third preferred embodiments, taken along line 2-2 of FIG. 25.

FIG. 25 is an enlarged transverse cross sectional view taken along line 2-2 of FIG. 24.

FIG. 26 is a view suitable for explaining a specific example of a treatment device when the high-frequency needle shown in FIG. 24 is used in the Brockenbrough method.

FIG. 27 is a vertical cross sectional view of the high-frequency needle shown in FIG. 24 projecting from a dilator.

FIG. 28 is a vertical cross sectional view of a distal end portion of a high-frequency needle as another practical embodiment (a sixth practical embodiment) of the present invention according to the fifteenth to twenty-third preferred embodiments.

FIG. 29 is a vertical cross sectional view of a distal end portion of a high-frequency needle as yet another practical embodiment (a seventh practical embodiment) of the present invention according to the fifteenth to twenty-third preferred embodiments.

### EMBODIMENTS FOR CARRYING OUT THE INVENTION

Hereinafter, first through fourth practical embodiments of the present invention will be described below with reference to the drawings.

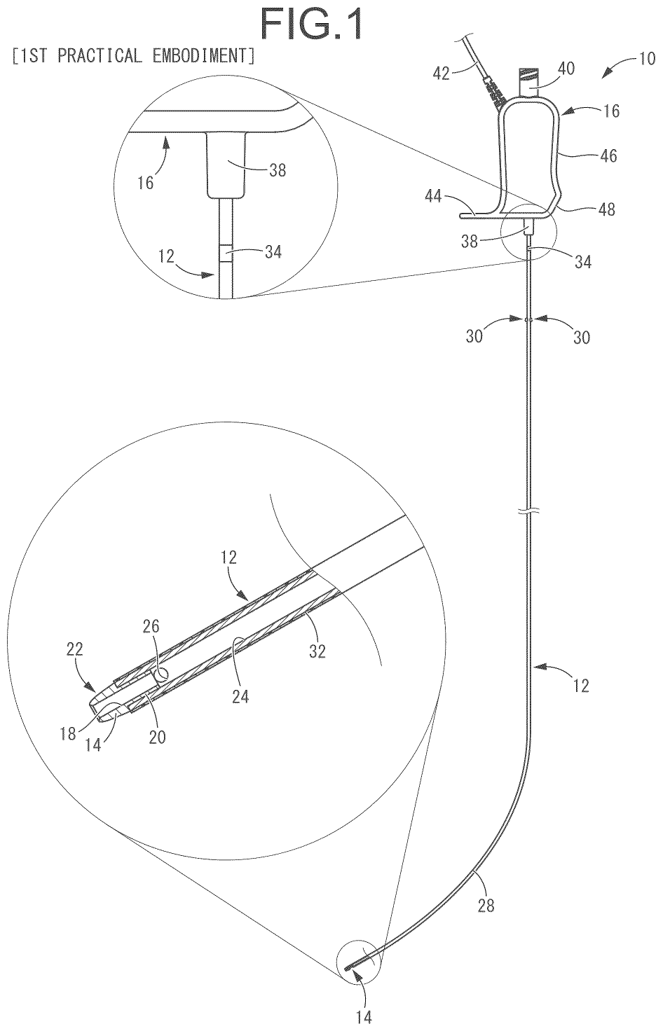

### First Practical Embodiment (FIGS. 1-18)

FIG. 1 shows a high-frequency needle 10 as a first practical embodiment of a perforation device according to the present invention. The high-frequency needle 10 has a structure including a shaft 12, a perforation head part 14 that is provided to the distal end of the shaft 12 and is configured to form a patent foramen in the body tissue, and an operation handle 16 that is provided to the proximal end of the shaft 12 and is configured to be gripped by a practitioner. In the following description, the proximal side refers to the proximal end side of the high-frequency needle 10 (the upper side in FIG. 1), which is the practitioner side in use, and the distal side refers to the distal end side of the high-frequency needle 10 (the lower side in FIG. 1), which is the patient side.

As shown enlarged in FIG. 1, the perforation head part 14 has an outer circumferential surface protruding from the distal end of the shaft 12 and exposed to the outside. The perforation head part 14 has a function of forming a patent foramen in the body tissue by energy supply from the outside. For example, the perforation head part 14 can cauterizes the body tissue by using the supplied high-frequency energy to form a patent foramen in the body tissue.

Besides, it is desirable that the perforation head part 14 be less permeable to X-rays (more radiopaque) than the shaft 12 described later. The perforation head part 14 of the present practical embodiment is formed of a metallic material such as gold, platinum, platinum iridium, tungsten, stainless steel, etc., which is highly visible under the X-ray fluoroscopy, and also functions as a distal end marker. A coating of radiopaque material can also be formed on the surface of the perforation head part 14 to ensure or improve its visibility under the X-ray fluoroscopy. The perforation head part 14 has a hollow structure penetrated by a through hole 18 in the length direction. The through hole 18 extends in the length direction with an approximately constant diameter, and its distal end portion is expanded toward the distal end.

The outer circumferential surface of the perforation head part 14 has a gradually decreasing diameter toward the distal end. The shape of the perforation head part 14 is not particularly limited, but it is desirable that the distal surface be a curved surface without corners to be less prone to stick when moving through the lumen. For example, the distal surface has an approximately semi-elliptical rotational shape that is convex toward the distal side, and has a warhead shape of an approximately round nose (a round head bullet) overall.

Besides, the perforation head part 14 includes a cylindrical connecting portion 20 extending in the axial direction from the proximal end side. By the connecting portion 20 being inserted and fastened to the distal end of the shaft 12, the perforation head part 14 is fixedly provided to the distal end of the shaft 12. Specifically, in the present practical embodiment, the perforation head part 14 is constituted as a distal end tip 22 integrally equipped with the connecting portion 20.

Such distal end tip 22 is provided with the through hole 18 extending continuously on the center axis between the perforation head part 14 and the connecting portion 20, so that the distal end tip 22 has an approximately cylindrical shape overall. The inner diameter dimension of the through hole 18 is smaller than the inner diameter dimension of the shaft 12, and the outer diameter dimension of the connecting portion 20 is smaller than the outer diameter dimension of the proximal end of the perforation head part 14, while being approximately the same as the inner diameter dimension of the shaft 12. The axial length of the connecting portion 20 is preferably longer than the axial length of the perforation head part 14, thereby improving fixing strength of the perforation head part 14 with respect to the shaft 12 and improving transmission efficiency of the feeling from the perforation head part 14 to the practitioner's hand.

The shaft 12 has a hollow tubular shape including a lumen 24 penetrating the shaft 12 in the length direction in the interior. The shaft 12 is flexible and deformable to follow the curve of somatic lumens such as a blood vessel. The shaft 12 is formed of conductive metal or other material, for example, a metal pipe. The peripheral wall face of the lumen 24 is constituted by the shaft 12, and for example, an electrically insulating protective layer may be provided on the radial inside of the shaft 12. The outer diameter dimension of the shaft 12 is approximately the same as the maximum outer diameter dimension (the proximal end outer diameter dimension) of the perforation head part 14, while the inner diameter dimension of the shaft 12 is approximately the same as the outer diameter dimension of the connecting portion 20. The shaft 12 can also be formed by, for example, applying conductive paste to a resin tube, or can be constituted by using conductive resin in which conductive filler is dispersed in the resin material.

The lumen 24 passes through the shaft 12 in the center axial direction, which is the length direction, and opens at the distal and proximal ends of the shaft 12. The lumen 24 has an approximately constant circular cross-section in the present practical embodiment, and extends in the length direction of the shaft 12, but the cross-sectional shape of the lumen 24 is not limited in particular. For example, a branch structure on the proximal end side or a structure with a varying cross-section in the center axis direction can also be adopted. The shaft 12 in the present practical embodiment has a single lumen structure including only one lumen 24, but the shaft 12 may, for example, have a multi-lumen structure including multiple lumens.

In the distal end portion of the shaft 12, side holes 26 are formed so as to penetrate a part of the peripheral wall and open onto the side circumferential surface (the outer circumferential surface). Each side hole 26 is a circular hole and extends to the lateral side so as to be approximately orthogonal to the center axis of the shaft 12, and communicates with the lumen 24. The side hole 26 of the present practical embodiment has a smaller diameter than that of the lumen 24. The side hole 26 is located away from the distal end of the shaft 12 toward the proximal end side. Two side holes 26 are formed on a straight line that passes through the shaft 12 in the diametrical direction, but there may be only one side hole 26, or three or more side holes 26 may be provided at a distance from one another in the circumferential direction. A plurality of side holes 26 may be provided at mutually different positions in the axial direction.

The distal end portion of the shaft 12 is provided with a shaft curved part 28 that is curved in one direction. The size of curvature, the length, and the change in curvature of the shaft curved part 28 are set appropriately according to the somatic lumen into which the shaft 12 is inserted. In the present practical embodiment, the shaft curved part 28 is set on the proximal end side with respect to the side holes 26. The shaft 12 can be plastically deformed by hand by the practitioner, and the practitioner may adjust the curvature, etc. of the shaft curved part 28 as necessary.

The proximal end portion of the shaft 12 is provided with a pair of first positioning protrusions 30, 30 serving as a positioning part protruding on the outer circumferential surface. The first positioning protrusion 30 may be formed by providing a partial protrusion on the shaft 12 itself, or may be provided as a separate component by means of a member for forming the first positioning protrusion 30 being fastened on the outer circumferential surface of the shaft 12 by clinching or the like. The pair of first positioning protrusions 30, 30 are provided, for example, at the same position in the axial direction so as to protrude toward the opposite sides in the radial direction.

The surface of the shaft 12 is covered by a coating layer 32. The coating layer 32 is formed by an electrically insulating resin coating, etc. For example, fluororesin (such as polytetrafluoroethylene (PTFE) with functional group substituted) is suitably employed as the coating material. The surface of the shaft 12 is covered by an electrically insulating coating material, whereby it is possible to avoid troubles such as loss of energy due to electric leakage and electric shock when the perforation head part 14 is supplied with electric power to perform perforation operation, for example. In particular, since the insulating layer is formed by the coating layer 32, the insulating layer can be made thin, thereby allowing the shaft 12 covered by the coating layer 32 to have a small diameter and minimizing the influence of the insulating layer on the curving deformation characteristics or the like of the shaft 12. Although the coating layer 32 is much thinner than the shaft 12, the thickness is exaggerated in the drawing for illustrative purposes.

As shown enlarged in FIG. 1, the coating layer 32 has a marker part 34 whose color is different from that of the other portions only at a part of the proximal end portion. The marker part 34 is annularly provided on a part of the axially proximal end portion of the shaft 12, for example. The color of the marker part 34 is not particularly limited, but a color that is easily distinguishable visually from the other portions of the coating layer 32, such as a yellow marker part 34 on a black coating layer 32, is selected as appropriate. The term “different color” does not only refer to differences in hue, but also includes differences in brightness or saturation. Therefore, even if the marker part 34 has the same hue as the other portions of the coating layer 32, it is acceptable as long as the marker part 34 is clearly distinguishable visually due to differences in brightness or saturation.

The specific structure and forming embodiment of the marker part 34 is not limited. For example, the marker part 34 can be formed with a coating film by applying a resin material or the like so as to cover the surface of the shaft 12. It would also be possible to form the marker part 34 by adhering a tube-shaped resin material of a different color from that of the shaft 12 to the shaft 12 as an outer layer. It would also be acceptable to form a transparent coating layer 32 on the surface of the shaft 12 that has been partially discolored by laser beam irradiation, etc., so that the discolored portion of the shaft 12 that is visible through the coating layer 32 may constitute the marker part 34. Alternatively, if the electric insulation properties of the outer circumferential surface of the shaft 12 is not required for the marker part 34, the coating layer 32 may be partially omitted to expose the surface of the shaft 12 to provide the marker part 34 of a different color from the coating layer 32. Besides, it is acceptable as long as the marker part 34 has an embodiment that exhibits alignment function of the shaft 12 with respect to a dilator 62 described later in the axial direction. In particular, the proximal end side position of the marker part 34 is not limited, but for example, the marker part 34 may extend to the proximal end side so as to reach the distal end of a distal end connecting part 38 of the operation handle 16 (the proximal end of the shaft 12). Furthermore, the color of the marker part 34 may be varied in multiple colors in the length direction so that the degree to which the distal end of the shaft 12 is approaching the distal end opening of the dilator 62 can be grasped by the difference in the visible color of the marker part 34.

In the present practical embodiment, the marker part 34 of annular shape extending over the entire circumference of the shaft 12 is exemplified, but the specific shape of the marker part is not particularly limited. For example, the marker part may be a line shape, such as a semicircular ring shape provided only on the upper half of the shaft 12, an arrow, a circle, a polygon, or any other shape that enables visual alignment in the axial direction of the shaft 12 with respect to the dilator 62 described later.

In a state where the perforation head part 14 protrudes from the distal end of the dilator 62 described later, the marker part 34 is housed in the dilator 62 and is not visible. Meanwhile, in a state where the perforation head part 14 is housed in the dilator 62, the marker part 34 is exposed to the proximal end side with respect to the dilator 62. Therefore, the practitioner can confirm that the perforation head part 14 is housed in the dilator 62 without protruding from the distal end of the dilator 62 by visually observing the marker part 34, which is exposed from the proximal end of the dilator 62 and visible from the outside. In particular, by aligning the marker part 34 with the proximal end of the dilator 62 and making the marker part 34 visible at a position adjacent to the proximal end of the dilator 62 in the axial direction, the perforation head part 14 is housed in the dilator 62 without being significantly remote from the distal end of the dilator 62 and without protruding from the dilator 62 to the distal end side. That is, the marker part 34 functions as a mark to indicate that the perforation head part 14 is properly housed in the distal end portion of the dilator 62 by being aligned with the proximal end of the dilator 62.

It is desirable that the marker part 34 visibly indicate that the perforation head part 14 has been housed in the dilator 62 in a state where the distance L from the distal end of the perforation head part 14 to the distal end of the dilator 62 is 20 mm or less, more preferably 10 mm or less. For example, when the perforation head part 14 is moved to the proximal side and retracted into the dilator 62 to be housed therein, before the distance L, which indicates the retraction amount of the distal end of the perforation head part 14 into the dilator 62, exceeds 20 mm, more preferably 10 mm, the marker part 34 is exposed to the proximal end side of the dilator 62 and visibly indicate that the perforation head part 14 has been housed in the dilator 62. This prevents the perforation head part 14 from being significantly retracted to the proximal side with respect to the dilator 62 and allows the perforation head part 14 to be housed in the distal end portion of the dilator 62.

The distal end of the marker part 34 is provided at the proximal end portion of the shaft 12, suitably within 20 mm, more suitably within 10 mm from the distal end of the operation handle 16 provided on the proximal end side of the shaft 12. The axial length of the marker part 34 is suitably 1 mm or more, more suitably 3 mm or more, thereby facilitating the formation of the marker part 34 and ensuring good visibility at the marker part 34.

The coating layer 32 with the marker part 34 of a different color can be formed, for example, as follows. That is, after forming the coating layer 32 other than the marker part 34 by applying a primary coating while masking the portion of the shaft 12 where the marker part 34 is to be formed, a secondary coating is applied to the masked portion with a coating material of a different color from that of the primary coating, thereby forming the marker part 34. This makes it possible to form the marker part 34 regardless of the material of the coating material (surface slipperiness, etc.), and to prevent the degree of freedom in selecting the coating material from being limited in order to form the marker part 34.

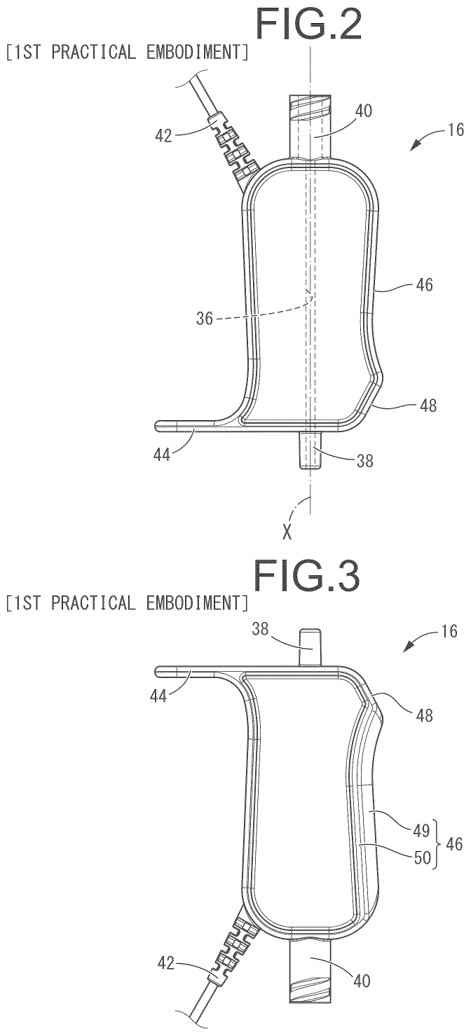

As shown in FIGS. 1 to 7, the operation handle 16 has an elongated plate shape whose axial length dimension (the vertical dimension in FIG. 2) is larger than its width dimension (the left-right dimension in FIG. 2). As shown in FIG. 7, the radially outer end of the operation handle 16 includes a rib-shaped part 35 protruding to the opposite sides in the plate thickness direction, so as to be thicker than the radially inner portion. In other words, lightening recesses are formed on the opposite sides of the operation handle 16 in the plate thickness direction. With this configuration, regarding the operation handle 16, reduction in the forming material and lighter weight is achieved, while the outer circumferential surface of the operation handle 16 reliably obtains a large area obtained by the rib-shaped part 35, which enables stable gripping by, for example, an embodiment of gripping the outer circumferential surface with a fingertip. The edge of the outer peripheral portion of the operation handle 16 is rounded to provide a good feel when held in the hand, and the edge portion is less likely to get caught on the surface of the hand or the like. Inside the operation handle 16, provided is an internal flow path 36 that communicates with the lumen 24 of the shaft 12. As shown in FIG. 2 in the dashed line for example, one end of the internal flow path 36 opens onto the distal end face of the operation handle 16 and communicates with the lumen 24, while the other end thereof opens onto the proximal end surface of the operation handle 16. A plurality of internal flow paths 36 can be provided in accordance with the lumen 24, and for example, the proximal end side may be branched into a plurality of flow paths.

As shown in FIGS. 2 to 4, a tubular distal end connecting part 38 protrudes from the distal end surface of the operation handle 16, and is fastened to the proximal end portion of the shaft 12. The distal end connecting part 38 is externally fastened to the proximal end of the shaft 12. The protruding length of the distal end connecting part 38 is not particularly limited, but is suitably set in the range of 5 to 10 mm. The internal flow path 36 of the operation handle 16 is connected to the lumen 24 of the shaft 12 inside the distal end connecting part 38.

As shown in FIGS. 2, 3, and 5, a tubular proximal end port part 40 protrudes from the proximal end surface of the operation handle 16. The proximal end port part 40 has a center hole communicating with the internal flow path 36, and the internal flow path 36 opens to the proximal end side via the proximal end port part 40. The outer circumferential surface of the proximal end port part 40 has a thread that is screwed with a female connector, for example. By connecting a three-way stopcock (not shown) or the like to the proximal end port part 40, the flow path connected to the lumen 24 can be branched into multiple paths, and ports for the administration of, for example, drug solutions, physiological saline, contrast media, etc., can be provided in appropriate combinations.

The operation handle 16 is provided with wiring 42 that is conductive with respect to the shaft 12. The wiring 42 extends to the proximal end side of the operation handle 16 at a position off the proximal end port part 40 and is connected to a power supply device (not shown). Electric power from the power supply device is supplied to the perforation head part 14 via the wiring 42 and the shaft 12 to activate the perforation head part 14 for perforation operation.

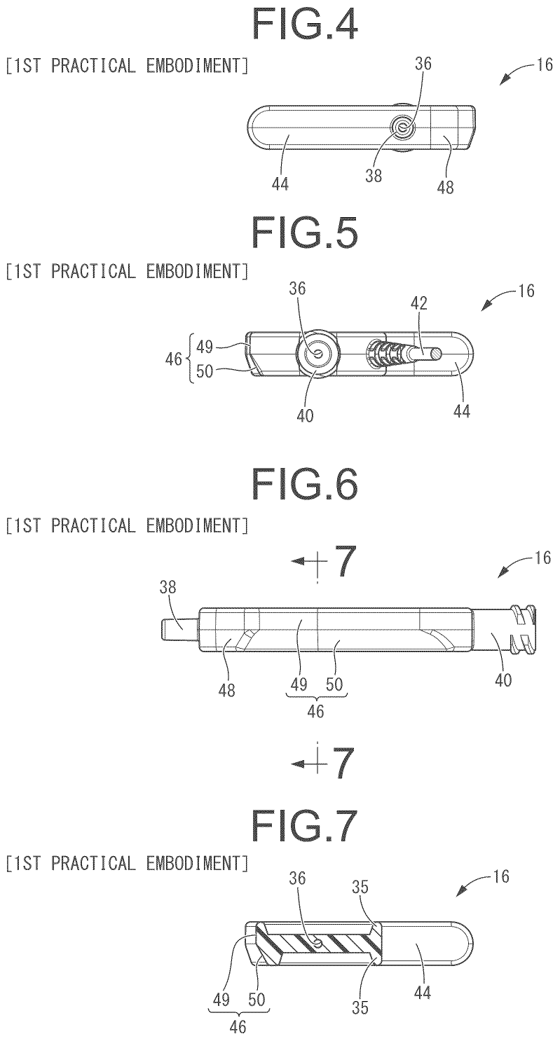

The plate thickness direction of the operation handle 16 (the vertical direction in FIG. 4) is approximately orthogonal to the plane including the shaft curved part 28. A curve indicator projection 44 protruding in the direction of curving of the shaft curved part 28 is integrally formed with the distal end part of the operation handle 16. As shown in FIGS. 2, 5, and 7, the curve indicator projection 44 has a plate shape extending approximately orthogonally to the axial direction of the operation handle 16 (the vertical direction in FIG. 2), and is elongated with the length dimension in the direction of protrusion being larger than the width dimension. The direction of curving of the shaft curved part 28 can be confirmed at the operation handle 16 by the practitioner visually or tactilely grasping the direction of protrusion of the curve indicator projection 44 protruding from the outer circumferential surface of the operation handle 16.

Regarding the outer circumferential surface of the operation handle 16, the surface on the side opposite to the side of protrusion of the curve indicator projection 44 includes a proximal end concave surface 46 constituted by the proximal end portion and a distal end inclined surface 48 constituted by the distal end portion, as shown in FIGS. 2 and 3. The proximal end concave surface 46 is a curved surface having a gentle concave shape opening outward (toward the right side in FIG. 2) viewed in the plate thickness direction of the operation handle 16. The distal end inclined surface 48 is an inclined surface that is inclined inward in the width direction toward the distal end.

A gripping surface 49 is set on the proximal end concave surface 46, which constitutes the outer circumferential surface of the operation handle 16. As shown in FIGS. 5 to 7, the gripping surface 49 extends approximately parallel to the plate thickness direction of the operation handle 16. The gripping surface 49 is partially provided in the plate thickness direction of the operation handle 16, and for example, is provided over the approximately half of the plate thickness on the outer circumferential surface (the proximal end concave surface 46) of the operation handle 16.

The portion of the proximal end concave surface 46 adjacent to the gripping surface 49 in the plate thickness direction of the operation handle 16 constitutes a rotational operation assisting surface 50. As shown in FIGS. 3, 5 and 7, the rotational operation assisting surface 50 provided on the outer circumferential surface of the operation handle 16 extends so as to be inclined with respect to the plate thickness direction (the vertical direction in FIG. 5), and is inclined relative to the gripping surface 49. The rotational operation assisting surface 50 and the gripping surface 49 are inclined at an inclination angle of 120 degrees relative to each other. On the outer circumferential surface (the proximal end concave surface 46) of the operation handle 16, the rotational operation assisting surface 50 is partially provided in the plate thickness direction of the operation handle 16, and for example, is provided over the approximately half of the plate thickness. The gripping surface 49 and the rotational operation assisting surface 50 are provided on the outer circumferential surface of the operation handle 16 on the side opposite to the direction of curving of the shaft curved part 28.

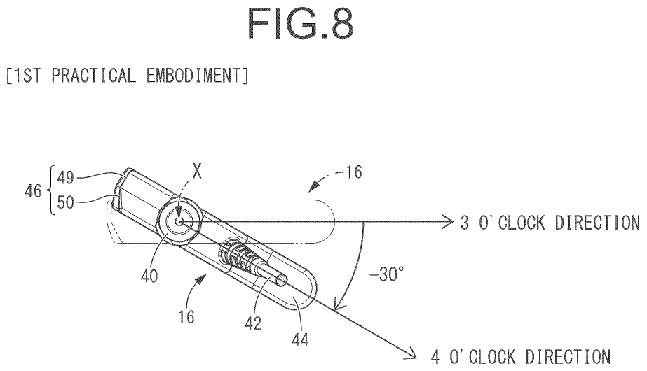

As shown in FIG. 8, viewed in the axial direction of the operation handle 16 from the proximal side to the distal side, when the operation handle 16 is rotated around the rotation center axis X (see FIG. 2) so that the direction of curving of the shaft curved part 28 is rotated approximately −30 degrees (approximately 30 degrees clockwise) from the horizontal plane, the rotational operation assisting surface 50 will extend to be approximately parallel to the vertical direction. In other words, by rotating the operation handle 16 around the rotation center axis X so that the rotational operation assisting surface 50 extends in the approximately vertical direction in the cross section orthogonal to the rotation center axis X (for example, the cross section corresponding to FIG. 7), the direction of curving of the shaft curved part 28 can be set to the direction rotated approximately −30 degrees from the horizontal plane. The operation of rotating the direction of curving of the shaft curved part 28 approximately −30 degrees from the horizontal plane is referred to as turning to the 4 o'clock direction. This is because, viewed in the axial direction of the operation handle 16 from the proximal side to the distal side, the direction of curving of the shaft curved part 28, which is the horizontal direction, is directed to the 3 o'clock direction, and the direction of curving of the shaft curved part 28 rotated approximately 30 degrees clockwise from the horizontal direction coincides with the 4 o'clock direction. For example, in a transseptal approach to form a patent foramen in the atrial septum, when the distal end of the shaft curved part 28 (the perforation head part 14) is turned to the fossa ovalis A of the atrial septum, it is necessary to turn the direction of curving of the shaft curved part 28 to the approximately 4 o'clock direction around the body axis parallel line (the rotation center axis X) of the patient. It is acceptable as long as the approximately 4 o'clock direction is a direction that does not significantly hamper the perforation position of the atrial septum in the transseptal approach procedure, and for such technical purposes, the angle is not strictly specified. Specifically, the approximately 4 o'clock direction includes, for example, embodiments in which the direction is set within the range of 3:30 to 5:30 direction depending on the site and purpose of treatment, etc. For normal perforation applications of the fossa ovalis, the direction is set within the range of 3:30 to 5 o'clock direction (−15 to −60 degrees from the horizontal plane around the body axis parallel line of the patient). The rotation center axis X of the operation handle 16 coincides with the center axis of the proximal end portion of the shaft 12 extending to the distal side from the operation handle 16.

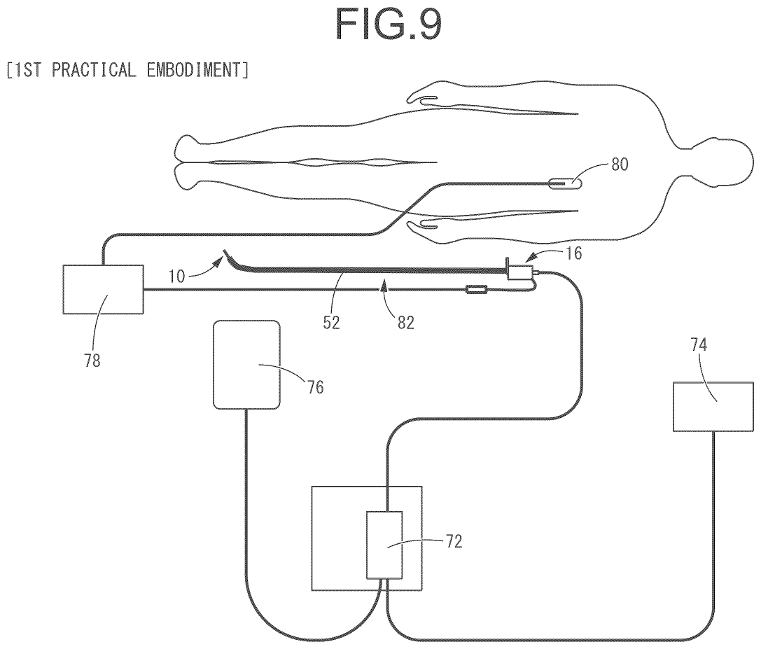

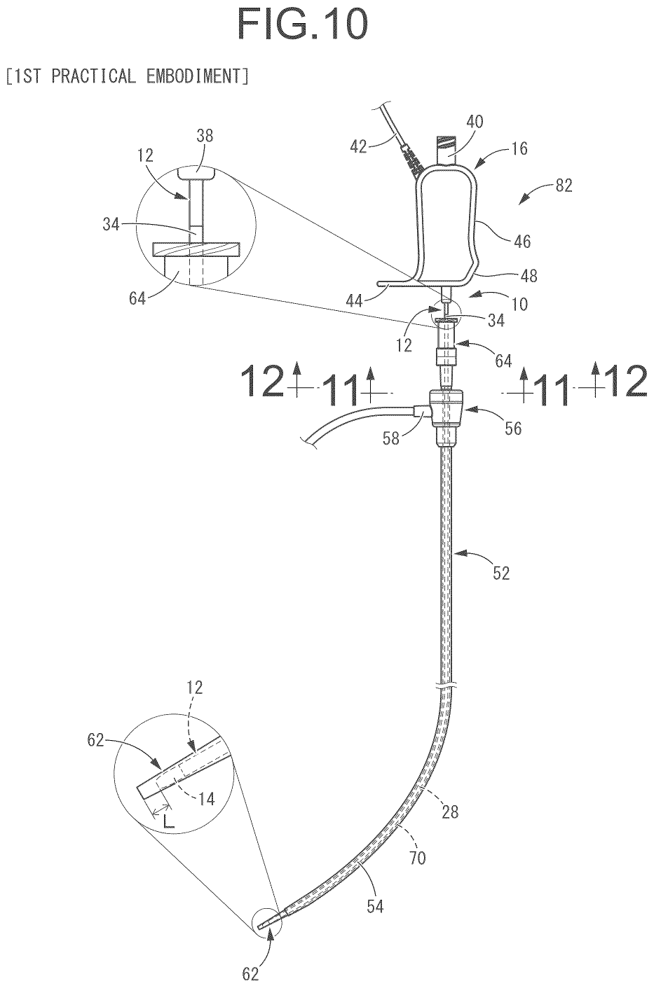

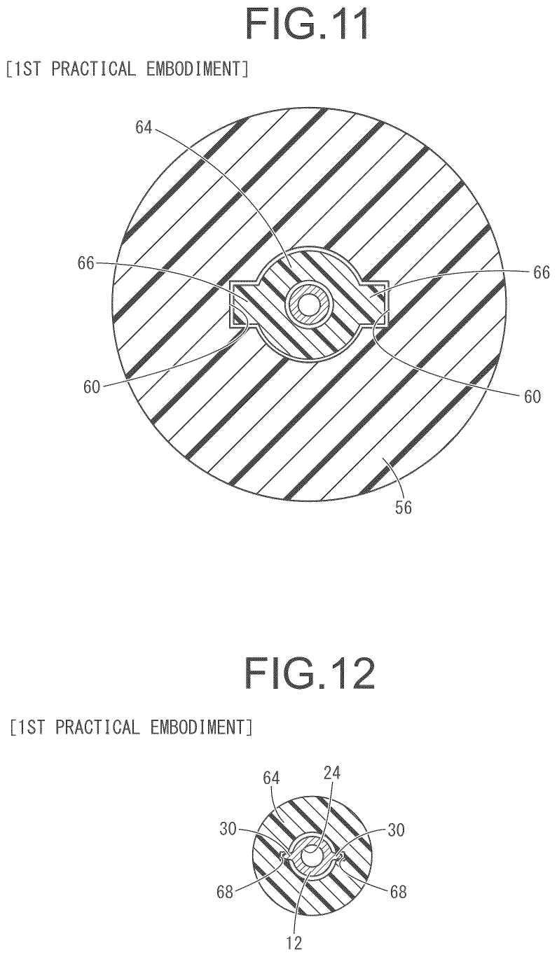

The high-frequency needle 10 with such a structure is used, for example, in a procedure to form a patent foramen in the atrial septum (the Brockenbrough method). The high-frequency needle 10 is inserted through a sheath 52, as shown in FIGS. 9 and 10 for example. The sheath 52 is flexible and tube-shaped, with a lumen penetrating in the length direction and opening at the distal end and the proximal end. The distal end portion of the sheath 52 constitutes a sheath curved part 54 to which a predetermined curved shape is set in advance. At the proximal end of the sheath 52, a rigid sheath hub 56 is provided, thereby allowing a contrast agent or the like to be administered to the distal end side of the sheath 52 through a side port 58 provided in the sheath hub 56. The sheath hub 56 includes second positioning recesses 60 opening onto its inner circumferential surface. In the present practical embodiment, a pair of second positioning recesses 60, 60 are formed on the opposite sides in the diametrical direction (see FIG. 11).

A medical device other than the high-frequency needle 10 may be inserted through the sheath 52 as needed. In the present practical embodiment, as shown in FIGS. 9 and 10, a dilator 62 is inserted through the sheath 52, and the shaft 12 of the high-frequency needle 10 is inserted through the dilator 62. In FIGS. 9 and 10, for illustrative purposes, the high-frequency needle 10 is shown inserted through the dilator 62, which has been inserted into the sheath 52 (dilator in sheath), outside the body. Indeed, the shaft 12 of the high-frequency needle 10 is to be inserted through the dilator 62, which has been arranged in the sheath 52, after the sheath 52 is inserted into the body by a guide wire (not shown). By the dilator 62 being inserted through the sheath 52 and the high-frequency needle 10 being inserted through the dilator 62, the high-frequency needle 10, the dilator 62, and the sheath 52 constitute a perforation device assembly 82, which will be described later.

The dilator 62 has a function for facilitating the insertion of the sheath 52 or the like through the patent foramen by being inserted into the patent foramen formed in the atrial septum by the high-frequency needle 10 thereby pushing open the patent foramen. The dilator 62 is made of resin, such as polyethylene, and has an approximately cylindrical shape, with the outer circumferential surface of at least the distal end portion gradually increasing in diameter from the distal end to the proximal end. Besides, a rigid dilator hub 64 is provided at the proximal end of the dilator 62. As shown in FIG. 11, the dilator hub 64 includes a pair of second positioning protrusions 66, 66, which correspond to the second positioning recesses 60, 60 of the sheath 52, protruding radially outward. As shown in FIG. 12, the dilator hub 64 includes a pair of first positioning recesses 68, 68, which correspond to the first positioning protrusions 30, 30 of the high-frequency needle 10, opening onto the inner circumferential surface.

As shown in FIG. 10, the distal end portion of the dilator 62 constitutes a dilator curved part 70 to which a predetermined curved shape is set in advance. The shaft curved part 28, the sheath curved part 54, and the dilator curved part 70 have mutually corresponding curved shapes, and the shaft curved part 28 can be inserted into the dilator curved part 70, and the dilator curved part 70 can be inserted into the sheath curved part 54. The radius of curvatures, the lengths, and the like of the shaft curved part 28, the sheath curved part 54, and the dilator curved part 70 are set appropriately according to the site to be treated, the type of procedure, and the like. The shaft curved part 28, the sheath curved part 54, and the dilator curved part 70 of the present practical embodiment are curved in a two-dimensional (planar) way toward one direction, but may be curved in a three-dimensional (solid) way toward multiple directions, for example.

As shown in FIG. 9, a transducer 72 is connected to the shaft 12 of the high-frequency needle 10. The transducer 72 converts the pressure in the lumen 24 of the shaft 12 into an electrical signal to display on a monitor 74. A pressurizing bag 76 containing physiological saline is connected to the lumen 24. The pressurizing bag 76 supplies physiological saline to the lumen 24 at a pressure equal to or greater than the blood pressure, thereby preventing blood from flowing through the lumen 24 into the transducer 72. Pressurization of the lumen 24 by the pressurizing bag 76 can be controlled, for example, according to the intracardiac pressure acting in the lumen 24. In the present practical embodiment, by detecting changes in the pressure in the lumen 24, it is possible to grasp the state of perforation, such as before, during, and after the perforation (after insertion into the left atrium).

A high frequency generator 78 is connected to the shaft 12 of the high-frequency needle 10 via the wiring 42. High-frequency energy generated by the high frequency generator 78 is supplied to the perforation head part 14 through the shaft 12, causing the perforation head part 14 to perform perforation operation. A counter electrode plate 80 connected to the high frequency generator 78 is attached to the patient to prevent electric shock to the patient during the high frequency energization.

Since the Brockenbrough method is conventionally known, a detailed description will be omitted. First, the practitioner inserts the sheath 52, into which the dilator 62 is inserted, from the inferior vena cava to the superior vena cava via the right atrium along a guide wire that has been previously inserted through a somatic lumen, such as a blood vessel.

As shown in FIG. 11, regarding the dilator 62 and the sheath 52, the second positioning protrusions 66 are inserted in the second positioning recesses 60, and the dilator 62 and the sheath 52 are mutually positioned in the circumferential direction and the amount of relative rotation is limited by the circumferential engagement of the second positioning protrusions 66 and the second positioning recesses 60. In this way, in the present practical embodiment, a second circumferential positioning mechanism for mutually positioning the dilator 62 and the sheath 52 in the circumferential direction is constituted by the circumferential engagement of the second positioning protrusions 66 and the second positioning recesses 60. Besides, the dilator 62 and the sheath 52 may be mutually positioned in the axial direction in an embodiment that can be easily released.

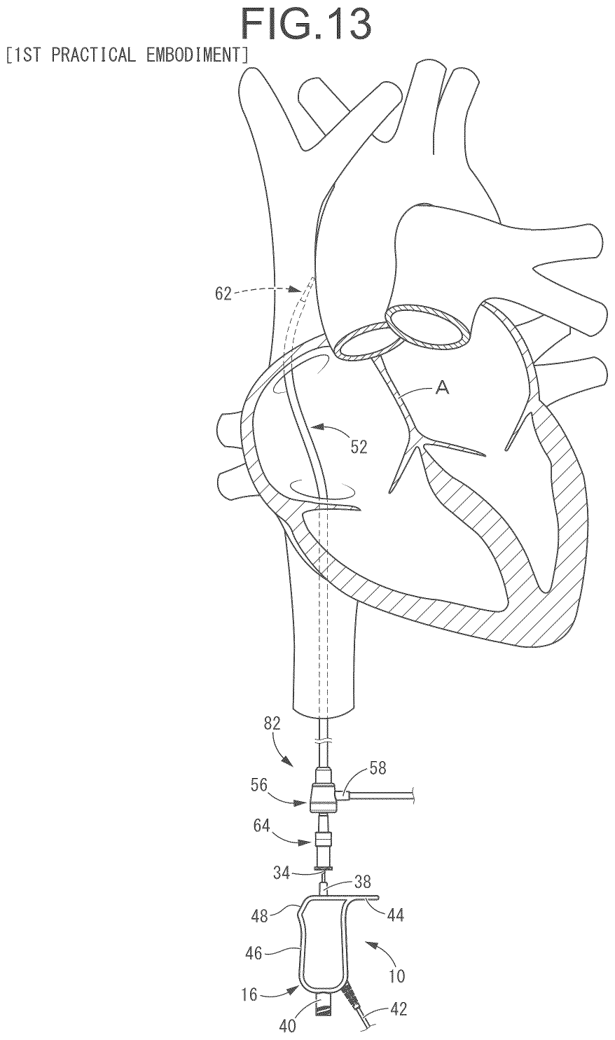

Next, as shown in FIG. 13, the practitioner inserts the high-frequency needle 10 through the dilator 62, which has been inserted into the sheath 52, and into the superior vena cava, so as to constitute the perforation device assembly 82. Regarding the perforation device assembly 82, at least the central axis at the proximal end portion is a body axis parallel line that is approximately parallel to the body axis of the patient. Note that the guide wire is removed prior to the insertion of the high-frequency needle 10.

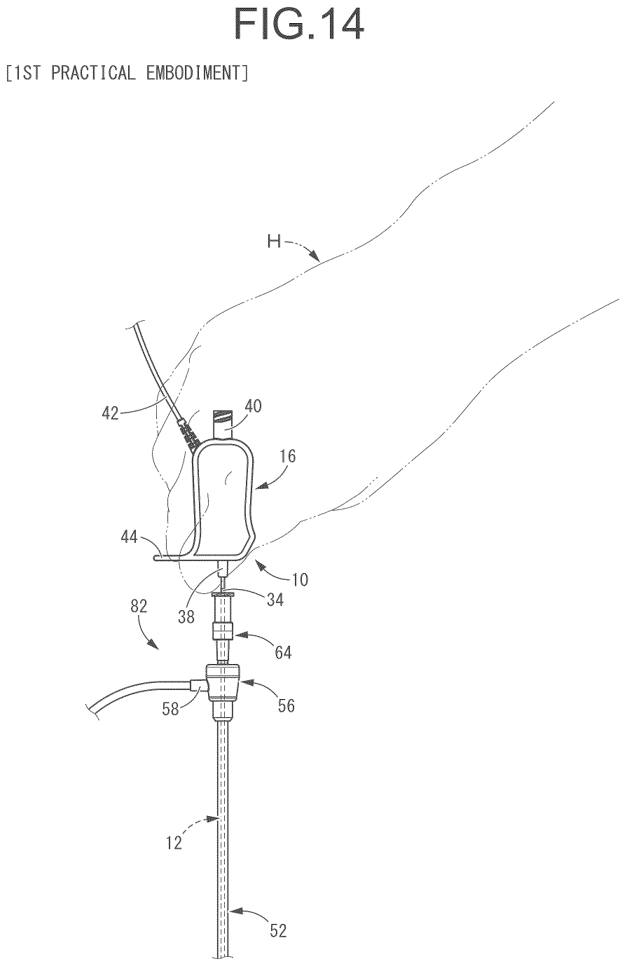

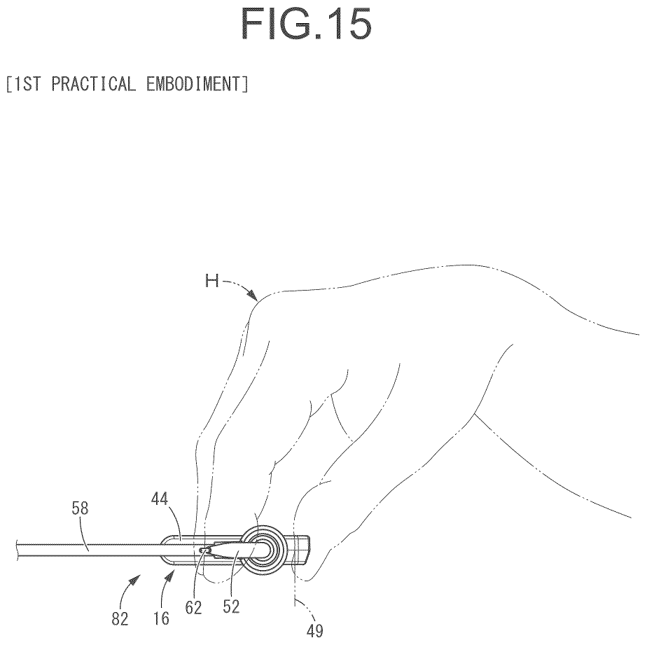

When inserting the high-frequency needle 10 into the dilator 62, the practitioner grasps the operation handle 16 as shown in FIGS. 14 and 15, and holds the perforation head part 14 directed to the 3 o'clock direction. In the present practical embodiment, the operation handle 16 is plate-shaped, and the plate thickness direction of the operation handle 16 and the plane including the central axis of the shaft curved part 28 are approximately orthogonal. Thus, the direction of curving of the shaft curved part 28 (the orientation of the perforation head part 14) can be grasped and held based on the plate thickness direction of the operation handle 16 or the like.

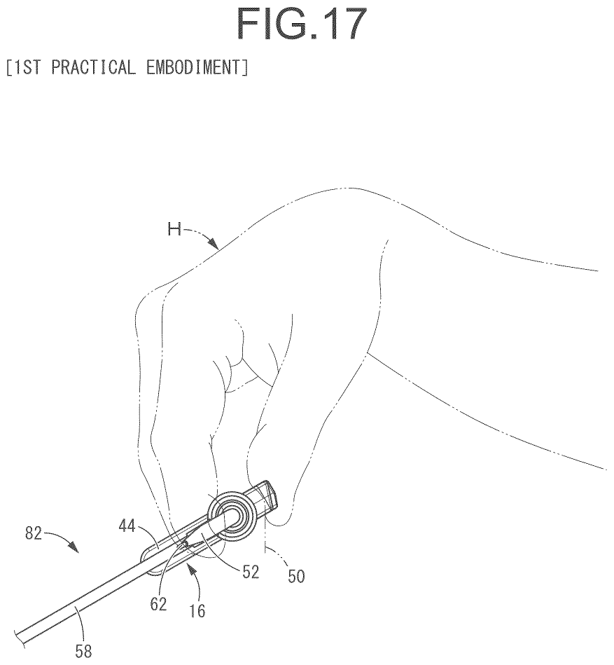

In particular, with the perforation head part 14 directed to the 3 o'clock direction, the plate thickness direction of the operation handle 16 coincides with the vertical direction, and the outer circumferential surface of the operation handle 16, including the gripping surface 49, is approximately parallel to the plate thickness direction of the operation handle 16, except for the rotational operation assisting surface 50. Thus, the perforation head part 14 is easily held to be directed to the 3 o'clock direction. Therefore, when inserting the high-frequency needle 10 into the dilator 62, by holding the gripping surface 49, not the rotational operation assisting surface 50, so as to grip with the fingertips, it is easy to hold the operation handle 16 so that the plate thickness direction approximately coincides with the vertical direction and the perforation head part 14 is directed to the 3 o'clock direction. In FIGS. 14 to 17, the hand H of the practitioner gripping the operation handle 16 is hypothetically shown in the chain double-dashed line. Besides, in FIGS. 15 and 17, the drawing symbol of one of the gripping surface 49 and the rotational operation assisting surface 50, which the thumb of the practitioner's hand H touches, is assigned to the extension line of the contact area of the thumb with those surfaces 49 and 50.

In particular, the input to the gripping surface 49 acts on the operation handle 16 in a direction approximately orthogonal to the plate thickness direction. This makes it easier to prevent the operation handle 16 from being accidentally rotated by the force acting thereon due to holding its outer circumferential surface.

Next, the practitioner pulls the operation handle 16 to the proximal side with respect to the dilator 62 to align the marker part 34 of the shaft 12 with the proximal end of the dilator hub 64. This causes the perforation head part 14 to be housed in the dilator 62 without protruding from the distal end of the dilator 62. Therefore, the practitioner can easily confirm that the perforation head part 14 inserted into the body is housed without protruding from the dilator 62 by visually checking the marker part 34 provided on the proximal end side of the shaft 12 outside the body. The perforation device assembly 82 shown in FIG. 10 is partially enlarged to show that the marker part 34 is aligned with the proximal end of the dilator hub 64, and the perforation head part 14 is housed in the dilator 62.

In particular, in the present practical embodiment, the alignment of the marker part 34 with the proximal end of the dilator hub 64 causes the perforation head part 14 to be housed in the dilator 62 with the distal end of the perforation head part 14 being located within 20 mm from the distal end of the dilator 62. By preventing the perforation head part 14 from being retracted to a position that is significantly remote from the distal end of the dilator 62, the shaft curved part 28, the dilator curved part 70, and the sheath curved part 54 are prevented from shifting significantly in the length direction. This can inhibit the dilator curved part 70 and the sheath curved part 54 from being deformed by the shaft curved part 28.

By aligning the marker part 34 with the proximal end of the dilator hub 64 when inserting the high-frequency needle 10 into the dilator 62, the perforation head part 14 can be properly housed in the dilator 62 in advance without pulling the high-frequency needle 10 to the proximal side after insertion into the dilator 62.

With the perforation head part 14 housed in the dilator 62, as shown in FIG. 12, the first positioning protrusions 30 of the shaft 12 are inserted into the first positioning recesses 68 of the dilator 62. By circumferential engagement of the first positioning protrusions 30 and the first positioning recesses 68, the high-frequency needle 10 and the dilator 62 are circumferentially positioned to limit the amount of relative rotation. In this way, in the present practical embodiment, a first circumferential positioning mechanism for mutually positioning the shaft 12 of the high-frequency needle 10 and the dilator 62 in the circumferential direction is constituted by the circumferential engagement of the first positioning protrusions 30 and the first positioning recesses 68.

The first circumferential positioning mechanism and the second circumferential positioning mechanism position the shaft 12 of the high-frequency needle 10, the dilator 62, and the sheath 52 in the circumferential direction. Thus, the shaft 12, the dilator 62 and the sheath 52 can be mutually combined in an appropriate circumferential orientation.

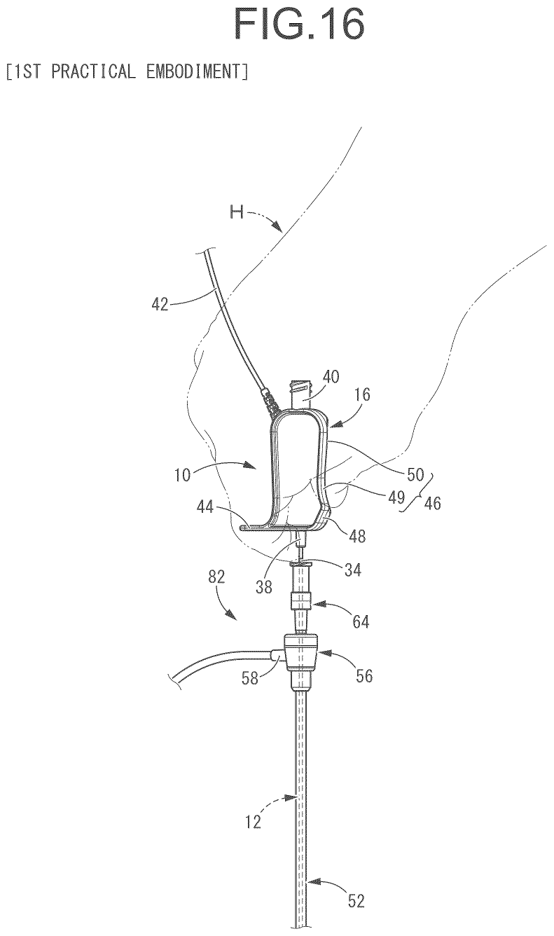

Next, as shown in FIGS. 16 and 17, the practitioner rotates the operation handle 16 to turn the direction of curving of the shaft curved part 28 to the approximately 4 o'clock direction around the body axis parallel line of the patient. At this time, the high-frequency needle 10 and the dilator 62 are circumferentially positioned by the first circumferential positioning mechanism, while the dilator 62 and the sheath 52 are circumferentially positioned by the second circumferential positioning mechanism. Thus, due to the rotation operation of the high-frequency needle 10, the dilator 62 and the sheath 52 rotate with the high-frequency needle 10 in an integrated manner. By so doing, the direction of curving of the dilator curved part 70 and the sheath curved part 54, together with the shaft curved part 28, are turned to the 4 o'clock direction, and the distal end of the perforation device assembly 82 is turned to the 4 o'clock direction. The integrated rotation of the perforation device assembly 82 is also realized by the mutual engagement of the curved parts 28, 70, and 54.

The circumferential positioning of the high-frequency needle 10, the dilator 62, and the sheath 52 is realized by the first circumferential positioning mechanism by means of circumferential engagement of the first positioning protrusions 30 and the first positioning recesses 68, and the second circumferential positioning mechanism by means of circumferential engagement of the second positioning protrusions 66 and the second positioning recesses 60, thereby obtaining the circumferential positioning with high reliability. This makes it possible to effectively prevent misorientation of the shaft 12, the dilator 62, and the sheath 52 due to their relative rotation at the insertion portion into the body, where direct visual confirmation is not possible.

Here, the operation handle 16 includes the rotational operation assisting surface 50 on its outer circumferential surface. By rotating the operation handle 16 so that the rotational operation assisting surface 50 extends vertically, the direction of curving of the shaft curved part 28, in other words, the direction of the distal end of the perforation head part 14, can be easily turned to the 4 o'clock direction. The rotational operation assisting surface 50 is provided on the operation handle 16, which is located outside the patient's body, and the practitioner can grasp visually or by hand feel whether the rotational operation assisting surface 50 extends vertically or not. Therefore, the direction of curving of the shaft curved part 28 (the orientation of the perforation head part 14), which is difficult to grasp when inserted into the body, can be easily and more reliably grasped. Also, by holding the operation handle 16 so that the rotational operation assisting surface 50 extends vertically, it is easy to continuously hold the orientation of the perforation head part 14 in the 4 o'clock direction.

Since the operation handle 16 is plate-shaped, it is possible to rotate the operation handle 16 or the like easily and accurately. Moreover, the rotational operation assisting surface 50 is provided as a relatively inclined surface that is not perpendicular or parallel to the plate thickness direction. Thus, it is easy to grasp the rotational operation assisting surface 50 by tactile feeling while gripping the operation handle 16.

On the outer circumferential surface of the operation handle 16, the rotational operation assisting surface 50 is located on the side opposite to the direction of curving of the shaft curved part 28. Therefore, the practitioner can operate the operation handle 16 while touching the rotational operation assisting surface 50 with the practitioner's thumb, which has an acute sense of touch, and the practitioner can easily grasp the state of the rotational operation assisting surface 50 extending vertically or horizontally.

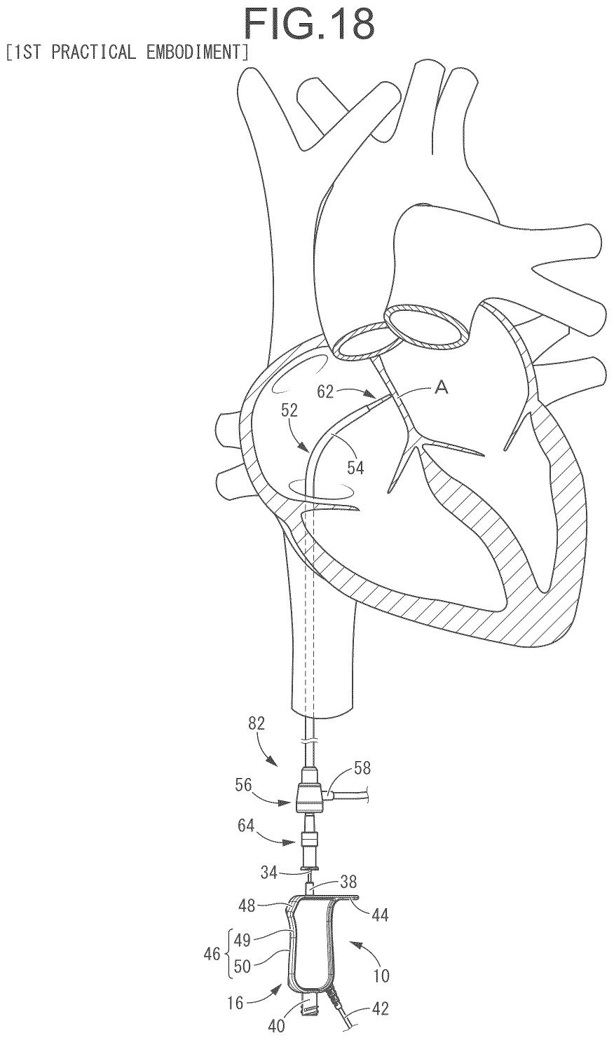

Next, as shown in FIG. 18, the practitioner pulls the perforation device assembly 82 to the proximal side to locate the distal end portion of the perforation device assembly 82 in the right atrium. The distal end portion of the perforation device assembly 82, which is turned to the 4 o'clock direction, is directed to the fossa ovalis A of the atrial septum separating the right atrium and the left atrium, and comes into contact with the fossa ovalis A. At this time, since the perforation head part 14 is housed in the dilator 62, damage to the body tissue including the fossa ovalis A is avoided, and the distal end parts of the flexible dilator 62 and the sheath 52 come into contact the body tissue such as the fossa ovalis A.

Next, the practitioner pushes the operation handle 16 to the distal side to advance the high-frequency needle 10 to the distal side with respect to the dilator 62. This causes the perforation head part 14 to protrude from the distal end of the dilator 62 and is pressed against the fossa ovalis A. The length of protrusion of the perforation head part 14 from the dilator 62 to the distal end is defined, for example, by the fit between the distal end connecting part 38 of the operation handle 16 and the proximal end opening of the dilator 62, which prevents excessive protrusion of the perforation head part 14. This prevents the perforation head part 14 of the high-frequency needle 10 from accidentally pressing strongly against the wall part of the left atrium, even if the high-frequency needle 10 protruding from the dilator 62 passes through the fossa ovalis A.

It would also be acceptable to advance the high-frequency needle 10 to the distal side with respect to the dilator 62 until the entire marker part 34 is housed in the dilator hub 64, so that the perforation head part 14 protrudes from the distal end of the dilator 62 by a predetermined length. In this case, the axial length of the marker part 34 is suitably 20 mm or less, more suitably 10 mm or less.

By supplying high-frequency electric power to the perforation head part 14, which is pressed against the fossa ovalis A, the perforation head part 14 performs perforation operation and forms a patent foramen in the fossa ovalis A. The high-frequency needle 10 is removed from the sheath 52 after forming the patent foramen in the fossa ovalis A. The dilator 62 is removed from the sheath 52 after the patent foramen is pushed open. The sheath 52 is inserted into the patent foramen dilated by the dilator 62, and a therapeutic device such as an ablation catheter is inserted into the left atrium through the sheath 52.

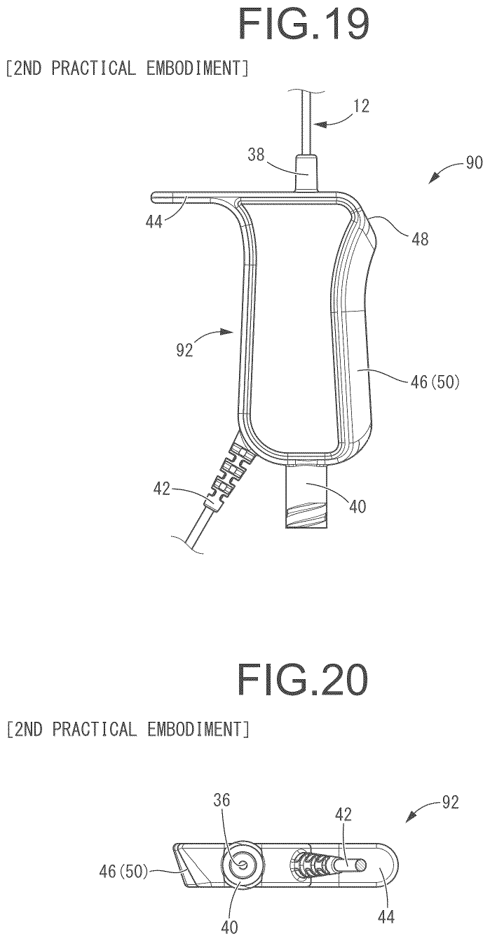

### Second Practical Embodiment (FIGS. 19-20)

FIGS. 19 and 20 show a proximal end portion of a high-frequency needle 90 as a second practical embodiment of a perforation device structured according to the present invention. In the following description, components and parts that are substantially identical with those in the first practical embodiment will be assigned like symbols and not described in any detail.

The high-frequency needle 90 has a structure in which an operation handle 92 is attached to the proximal end of the shaft 12. The operation handle 92 has a plate shape similar to that of the operation handle 16 of the first practical embodiment. In the operation handle 92, the proximal end concave surface 46 constitutes the rotational operation assisting surface 50 in its entirety, and the gripping surface 49 of the first embodiment is not provided.

Even with such a high-frequency needle 90 of the present practical embodiment, it is possible to hold the direction of curving of the shaft 12 in the 3 o'clock direction by paying attention to the direction and magnitude of the force applied to the operation handle 92, by gripping the operation handle 92 in a manner that is resistant to rotation, and the like.

When turning the direction of curving of the shaft 12 to the 4 o'clock direction, the rotational operation assisting surface 50 has a large area, which makes it easy to grasp the amount of rotational operation of the operation handle 92 by the rotational operation assisting surface 50. In particular, the proximal end concave surface 46 constitutes the rotational operation assisting surface 50 in its entirety. This reduces the risk of, for example, mistaking the amount of rotational operation by accidentally gripping a portion other than the rotational operation assisting surface 50.

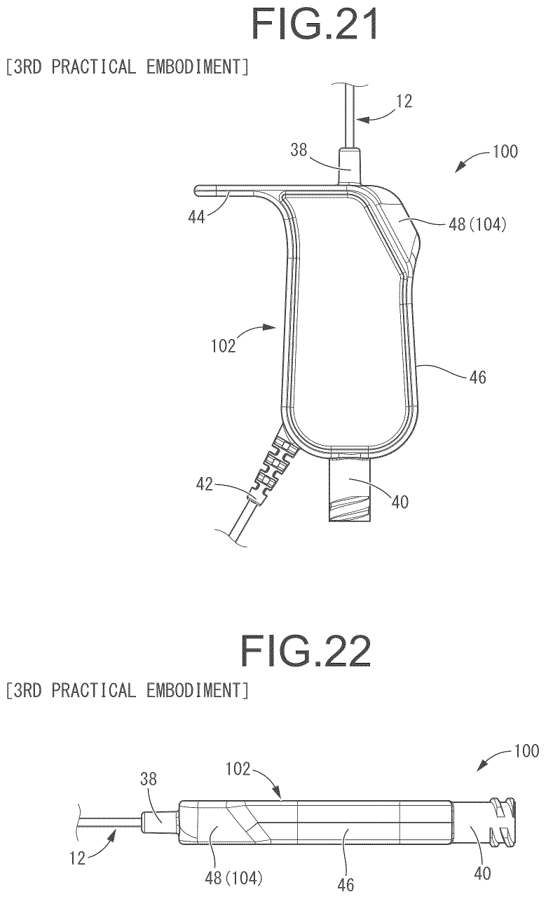

### Third Practical Embodiment (FIGS. 21-22)

FIGS. 21 and 22 show the proximal end portion of a high-frequency needle 100 as a third practical embodiment of a perforation device structured according to the present invention.

The high-frequency needle 100 has a structure in which an operation handle 102 is attached to the proximal end of the shaft 12. The operation handle 102 has a plate shape similar to that of the operation handle 16 of the first practical embodiment. Besides, a rotational operation assisting surface 104 is provided on the distal end inclined surface 48, which is a part of the outer circumferential surface of the shaft 12. Accordingly, the rotational operation assisting surface 104 is inclined in the plate thickness direction while being inclined with respect to the circumferential direction of the operation handle 102.

According to the high-frequency needle 100 of the present practical embodiment, for example, when inserting the high-frequency needle 100 into the dilator (not shown), by holding the proximal end concave surface 46 of the operation handle 102 so as to grip with the fingertips, it is easy to hold the operation handle 16 so that the plate thickness direction coincides with the vertical direction and the perforation head part (not shown) is directed to the 3 o'clock direction. In particular, the input to the proximal end concave surface 46 acts in a direction approximately orthogonal to the plate thickness direction of the operation handle 16. This makes it possible to prevent the operation handle 16 from being accidentally rotated by the force acting thereon due to holding its outer circumferential surface.

When turning the direction of curving of the shaft 12 to the 4 o'clock direction, the operation to turn it to the 4 o'clock direction can be easily performed by rotating the operation handle 102 while touching the rotational operation assisting surface 104 with the fingertip so that the rotational operation assisting surface 104 extends in the approximately vertical direction. It would also be acceptable that the rotational operation assisting surface 104 of the present practical embodiment is partially provided in the thickness direction of the operation handle 102, and that the gripping surface 49 as in the preceding first practical embodiment is provided side by side with the rotational operation assisting surface 104.

While the present invention has been described in detail hereinabove in terms of the practical embodiments, the invention is not limited by the specific description thereof. For example, the operation handle 16 is not limited to have a plate shape. Specifically, the operation handle 16 may have a rod shape, such as a cylinder, for example. In addition, the surface of the operation handle may be provided with an anti-slip structure such as grooves, protrusions, grains, etc. to function as a slip stopper to improve operability.

The preceding practical embodiment illustrates the rotational operation assisting surface 50 provided such that by being oriented so as to extend in the vertical direction, the perforation head part 14 turns to the approximately 4 o'clock direction. However, for example, the rotational operation assisting surface may be provided such that by being oriented so as to extend in the horizontal direction, the perforation head part 14 turns to the approximately 4 o'clock direction.

The location of the rotational operation assisting surface 50 on the operation handle 16 is set appropriately according to the way the practitioner holds the operation handle 16 or the like. For example, the rotational operation assisting surface 50 is suitably provided on the outer circumferential surface of the plate-shaped operation handle 16, but may alternatively be provided on the surface in the plate thickness direction.

The rotational operation assisting surface 50 may be provided at multiple locations on the operation handle 16. With this configuration, for example, in the operation handle 16, which may be held in different ways by different practitioners, by the rotational operation assisting surface 50 being provided at each part that is touched by the corresponding practitioner's way of holding the handle, it is possible to deal with various ways of holding the handle. When a plurality of rotational operation assisting surfaces 50 are provided, those rotational operation assisting surfaces 50 may extend approximately parallel to each other, or may extend approximately orthogonally to each other.

The preceding practical embodiment exemplifies a structure in which the marker part 34 is provided in the coating layer 32 covering the surface of the shaft 12. However, for example, the marker part can also be configured utilizing the distal end portion of the operation handle.

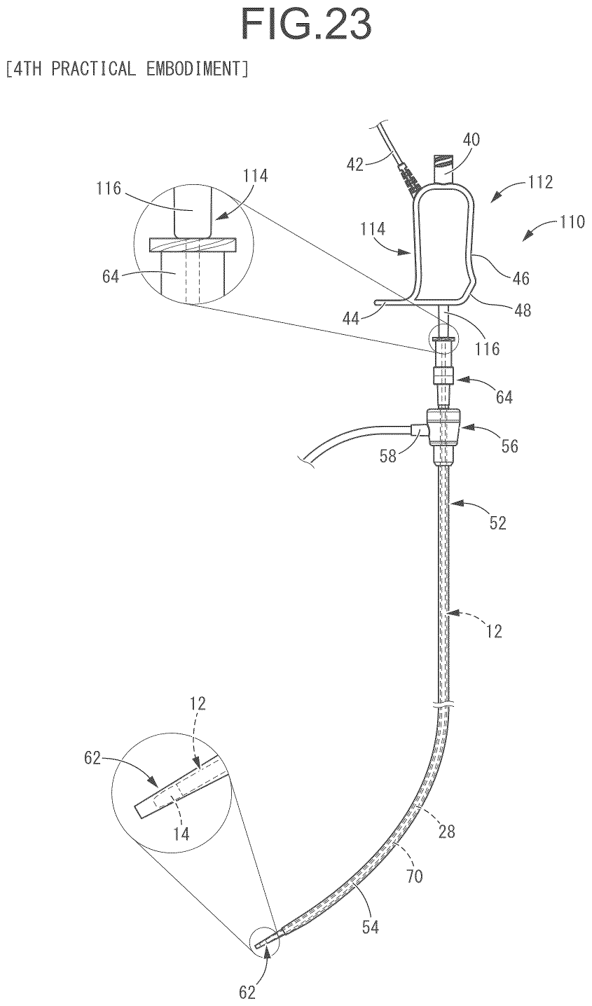

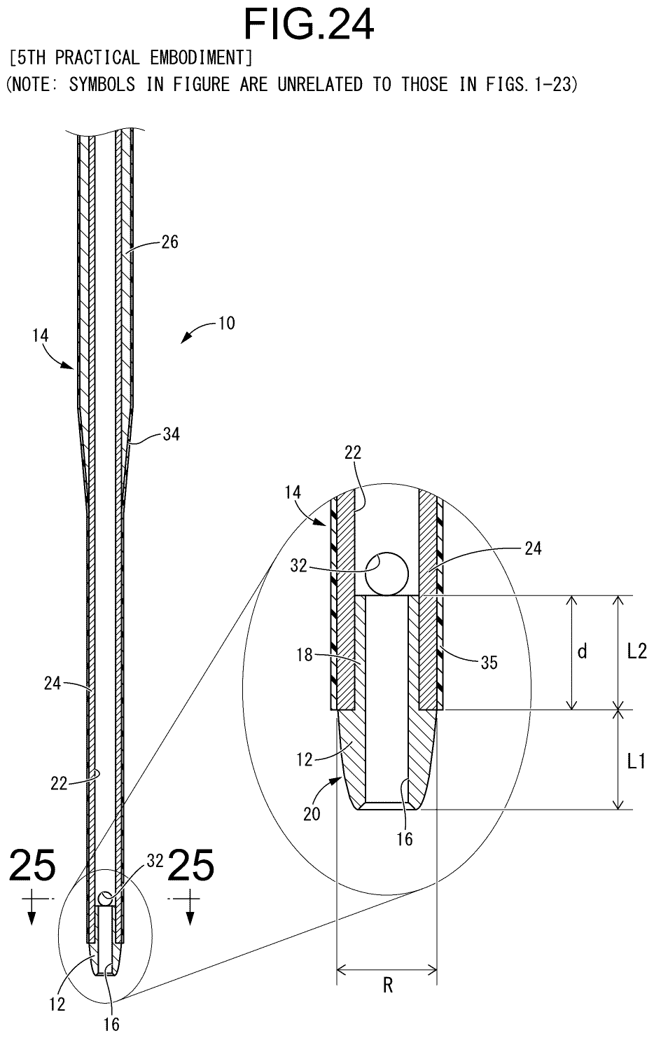

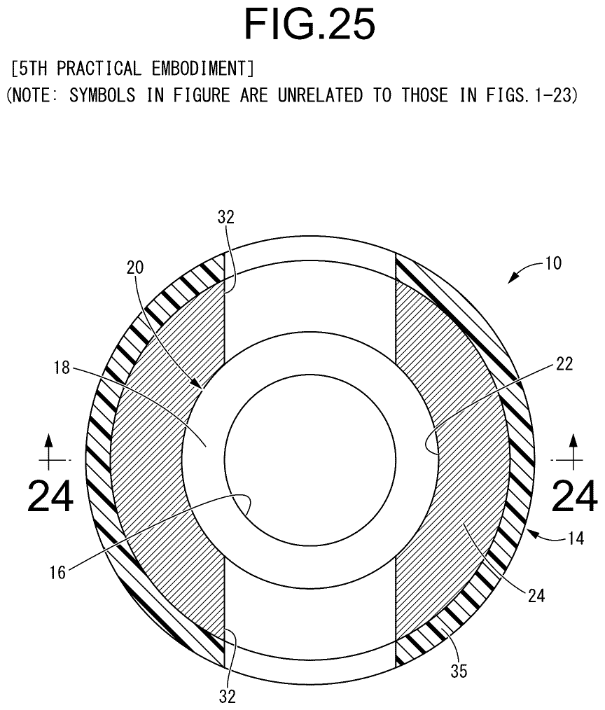

### Fourth Practical Embodiment (FIG. 23)

That is, in a perforation device assembly 110 shown in FIG. 23, an operation handle 114 constituting a high-frequency needle 112 serving as a perforation device includes a distal end connecting part 116 serving as an extended part extending out on the outer circumferential surface of the shaft 12. The distal end connecting part 116 is different in color from the color of the shaft 12 (the coating layer 32), and the position of the distal end of the distal end connecting part 116, which is the boundary between the distal end connecting part 116 and the shaft 12, can be easily confirmed visually. Then, for example, by aligning the distal end of the distal end connecting part 116 with the proximal end of the dilator hub 64, the perforation head part (not shown) is housed in the dilator 62 without protruding from the distal end of the dilator 62. Thus, in the present practical embodiment, the marker part is constituted by the distal end connecting part 116 of the operation handle 114. Besides, the lumen of the dilator hub 64 is configured to allow insertion of the distal end connecting part 116, and by pushing the distal end connecting part 116 distally to the position where it is inserted into the lumen of the dilator hub 64 from the proximal end side, the perforation head part can protrude from the distal end of the dilator 62 by an appropriate length. The distal end connecting part 116 may be inserted into the dilator hub 64 approximately in its entirety, or only the distal end portion thereof may be inserted into the dilator hub 64.

The preceding practical embodiment illustrates a marker part 34 that visibly indicates that the perforation head part 14 is housed in the dilator 62. However, for example, a mark (a marker) that visibly indicates that the perforation head part 14 protrudes from the distal end of the dilator 62 by an appropriate length, can be provided in the same way as the marker part 34. In the case where both the marker part 34, which indicates the housed state of the perforation head part 14, and the mark, which indicates the protruding state of the perforation head part 14, are provided, it is desirable that the marker part 34 and the mark be easily and surely distinguishable visually, for example, by using different colors for the marker part 34 and the mark, or the like.

The marker part 34 may be recognizable by the tactile sense of a finger. With this configuration, for example, the practitioner can move the perforation head part 14 in and out of the dilator 62 by the tactile sense of his/her fingers while watching the imaging monitor.

The preceding practical embodiment illustrates a high-frequency needle that performs perforation by means of high-frequency energy as an example of a perforation device. Alternatively, for example, it would also be possible to puncture the body tissue such as the atrial septum with a sharp hollow needle (e.g., a transseptal needle) to form a patent foramen. In this case, the shaft 12 does not need to be conductive, and can be formed of an insulating metal, resin, or the like. Besides, the shaft 12 does not need to have an insulating coating layer, and the marker part can be formed by, for example, making the shaft 12 partially different in color, providing partial painting or laser marking on the surface of the shaft 12, and so on.

The preceding practical embodiment illustrates an atrial septum perforation device, which is one type of a body tissue perforation device. However, the present invention can also be applied to perforation devices that form a patent foramen in a body tissue other than the atrial septum (the fossa ovalis). In particular, the marker part for indicating the position of the device relative to the dilator or the like in the length direction, and the positioning structure for positioning the perforation device, the dilator, and the sheath in the circumferential direction are not necessarily applicable only to perforation devices for the atrial septum.

Hereinafter, fifth through seventh practical embodiments of the present invention will be described below with reference to the drawings.

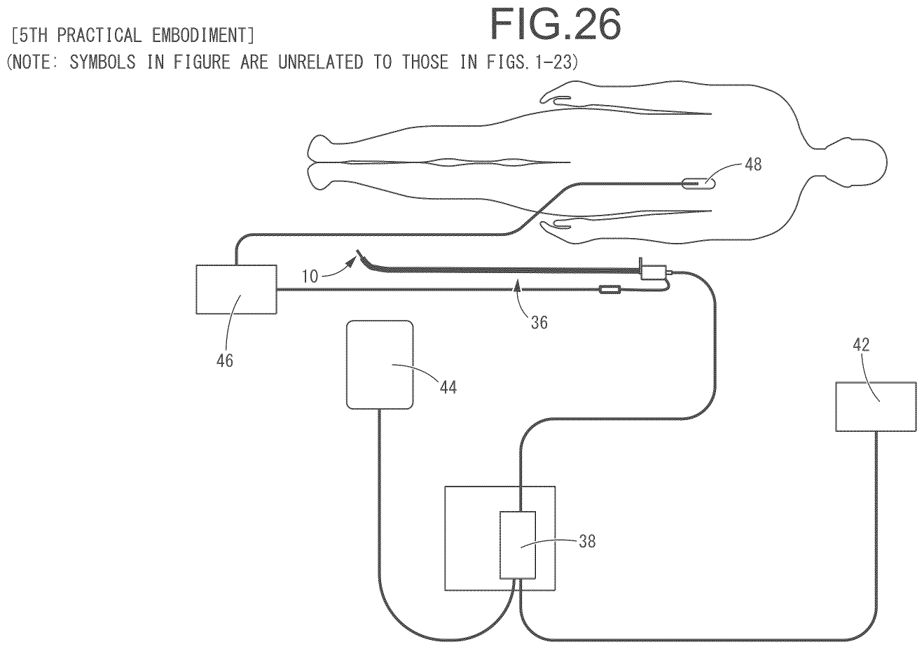

### [Fifth Practical Embodiment (FIGS. 24-27)] (Note: The Symbols in the Figures are Unrelated to Those in FIGS. 1-23)

FIGS. 24 and 25 show a high-frequency needle 10 as a first practical embodiment of a body tissue perforation device according to the present invention. The high-frequency needle 10 has a structure in which a perforation head part 12 is arranged on the distal end side of a tube 14. Hereinafter, the proximal end side of the high-frequency needle 10 (the upper side in FIG. 24), which is the practitioner side in use, will be described as the proximal end side, and the distal end side of the high-frequency needle 10 (the lower side in FIG. 24), which is the patient side, will be described as the distal end side.

The perforation head part 12 has an outer circumferential surface protruding from the distal end of the tube 14 and exposed to the outside. The perforation head part 12 has a function of forming a patent foramen in the body tissue by energy supply from the outside. For example, the perforation head part 12 can cauterizes the body tissue by using the supplied high-frequency energy to form a patent foramen in the body tissue.

Besides, it is desirable that the perforation head part 12 be less permeable to X-rays (more radiopaque) than an inner tube 24 described later. The perforation head part 12 of the present practical embodiment is formed of a metallic material such as gold, platinum, platinum iridium, tungsten, stainless steel, etc., which is highly visible under the X-ray fluoroscopy, and also functions as a distal end marker. A coating of radiopaque material can also be formed on the surface of the perforation head part 12 to ensure or improve its visibility under the X-ray fluoroscopy. The perforation head part 12 has a hollow structure penetrated by a through hole 16 in the length direction. The through hole 16 extends in the length direction with an approximately constant diameter, and its distal end portion is expanded toward the distal end.

The outer circumferential surface of the perforation head part 12 has a gradually decreasing diameter toward the distal end. The shape of the perforation head part 12 is not particularly limited, but it is desirable that the distal surface be a curved surface without corners to be less prone to stick when moving through the lumen. For example, the distal surface has an approximately semi-elliptical rotational shape that is convex toward the distal side, and has a warhead shape of an approximately round nose (a round head bullet) overall.

Besides, the perforation head part 12 includes a cylindrical connecting portion 18 extending in the axial direction from the proximal end side. By the connecting portion 18 being inserted and fastened to the distal end of the tube 14, the perforation head part 12 is fixedly provided to the distal end of the tube 14. Specifically, in the present practical embodiment, the perforation head part 12 is constituted as a distal end tip 20 integrally equipped with the connecting portion 18.

Such distal end tip 20 is provided with the through hole 16 extending continuously on the center axis between the perforation head part 12 and the connecting portion 18, so that the distal end tip 20 has an approximately cylindrical shape overall. The through hole 16 is smaller than the inner diameter dimension of the tube 14, and the outer diameter dimension of the connecting portion 18 is smaller than the outer diameter dimension of the proximal end of the perforation head part 12, while being approximately the same as the inner diameter dimension of the tube 14. The axial length of the connecting portion 18 is preferably longer than the axial length of the perforation head part 12, thereby improving fixing strength of the perforation head part 12 with respect to the tube 14 and improving transmission efficiency of the feeling from the perforation head part 12 to the practitioner's hand.

The perforation head part 12 has a maximum outer diameter dimension R set within the range of 0.5 to 1.0 mm, and more suitably, the maximum outer diameter dimension R is set within the range of 0.7 to 0.8 mm. With this configuration, while forming the through hole 16 of the required size (the inner diameter) in the perforation head part 12, good visibility of the perforation head part 12 under the X-ray fluoroscopy can be reliably obtained, and insertability into a dilator 40 (described later) and the somatic lumen can be improved.

The length dimension L1 of the perforation head part 12 is set within the range of 0.25 to 3.0 mm, suitably within the range of 0.25 to 1.5 mm, and also suitably within the range of 0.3 to 1.0 mm. With this configuration, insertability of the perforation head part 12 into the dilator 40 (described later) and the somatic lumen can be reliably obtained, and the high-frequency energy supplied during the perforation operation will be concentrated on the distal end portion of the perforation head part 12 to enable efficient perforation. In addition, by setting the length dimension L1 of the perforation head part 12 to 0.5 mm or more, good visibility of the perforation head part 12 under the X-ray fluoroscopy is realized.

When the connecting portion 18 is provided as in the present practical embodiment, the length dimension L2 of the connecting portion 18 is suitably set within the range of 0.3 to 2.5 mm from the viewpoint of compatibly securing the fixing force with respect to the tube 14 and improving the insertability into the somatic lumen (flexibility of the tube 14).

It is desirable that the perforation head part 12 have a peripheral wall portion that is continuous in the length direction for not less than 0.3 mm (or not less than 0.5 mm) with a radial thickness of not less than 0.1 mm. The peripheral wall portion of the present practical embodiment is set so that the perforation head part 12 and the connecting portion 18 constitute the peripheral wall of the through hole 16. The peripheral wall portion may be constituted only by the perforation head part 12. In that case, in the perforation head part 12, a peripheral wall thick portion, in which the radial thickness dimension of the peripheral wall of the through hole 16 is not less than 0.1 mm, is preferably continuous for not less than 0.3 mm (or not less than 0.5 mm) in the length direction. This will ensure the visibility of the peripheral wall portion (the perforation head part 12 and the connecting portion 18) that functions as the marker under the X-ray fluoroscopy. The peripheral wall portion may be constituted by the perforation head part 12 and the connecting portion 18 as described above, or may be constituted only by the perforation head part 12, or may alternatively be constituted only by the connecting portion 18 extending from the perforation head part 12 to the proximal end, for example.

The tube 14 has a hollow tubular shape including a lumen 22 penetrating the tube 14 in the length direction in the interior. The tube 14 is flexible and deformable to follow the curve of somatic lumens such as a blood vessel. The tube 14 includes an inner tube 24 and an outer tube 26 that is externally fastened to the inner tube 24.

The inner tube 24 is formed of conductive metal or other material, for example, a metal pipe. The peripheral wall face of the lumen 22 is constituted by the inner tube 24, and for example, an electrically insulating protective layer may be provided on the radial inside of the inner tube 24. The outer diameter dimension of the inner tube 24 is approximately the same as the maximum outer diameter dimension (the proximal end outer diameter dimension) of the perforation head part 12, while the inner diameter dimension of the inner tube 24 is approximately the same as the outer diameter dimension of the connecting portion 18. The inner tube 24 can also be formed by, for example, applying conductive paste to a resin tube, or can be constituted by using conductive resin in which conductive filler is dispersed in the resin material.

The lumen 22 passes through the inner tube 24 in the center axial direction, which is the length direction, and opens at the distal and proximal ends of the inner tube 24. The lumen 22 has an approximately constant circular cross-section in the present practical embodiment, and extends in the length direction of the inner tube 24, but the cross-sectional shape of the lumen 22 is not limited in particular. For example, a branch structure on the proximal end side or a structure with a varying cross-section in the center axis direction can also be adopted. The inner tube 24 in the present practical embodiment has a single lumen structure including only one lumen 22, but the inner tube 24 may, for example, have a multi-lumen structure including multiple lumens.

In the distal end portion of the inner tube 24, side holes 32 are formed so as to penetrate a part of the peripheral wall and open onto the side circumferential surface (the outer circumferential surface). Each side hole 32 is a circular hole and extends to the lateral side so as to be approximately orthogonal to the center axis of the inner tube 24, and communicates with the lumen 22. The side hole 32 of the present practical embodiment has a smaller diameter than that of the lumen 22. The side hole 32 is located away from the distal end of the inner tube 24 toward the proximal end side. The distance d from the distal end of the inner tube 24 to the opening distal end of the side hole 32 is not particularly limited, but is preferably in the range of 0.3 to 2.5 mm, for example. Two side holes 32 are provided in series in the circumferential direction of the inner tube 24, but there may be only one side hole 32, or three or more side holes 32 may be provided at a distance from one another in the circumferential direction.

The outer tube 26 is made of metal, for example, and is externally fitted onto the inner tube 24 at a portion away from the distal end of the inner tube 24 to the proximal end side, and is fixed to the inner tube 24 by means of adhesion or the like. The outer tube 26 is arranged on the proximal end side with respect to the side holes 32 of the inner tube 24. The distance from the proximal end of the opening of the side hole 32 to the distal end of the outer tube 26 is suitably longer than the distance d from the distal end of the inner tube 24 to the distal end of the opening of the side hole 32. The inner circumferential surface of the outer tube 26 is a cylindrical surface extending in the length direction with an approximately constant inner diameter dimension, and is overlapped on the outer circumferential surface of the inner tube 24. The outer circumferential surface of the outer tube 26 has a tapered surface 34 at its distal end portion that becomes larger in diameter as it moves away from the distal end. The outer circumferential surface of the outer tube 26 extends in the length direction with an approximately constant outer diameter dimension on the proximal end side with respect to the tapered surface 34. The tube 14 has a larger outer diameter dimension at the proximal end portion where the outer tube 26 is provided than at the distal end portion located at the distal end side with respect to the outer tube 26. In order to prevent excessive bending rigidity of the tube 14, the inner tube 24 may have a slit, a lightening part, or the like at the portion where the outer tube 26 is externally fitted.

The surfaces of the inner tube 24 and the outer tube 26 are covered by a coating layer 35. The coating layer 35 is formed by an electrically insulating resin coating, etc. Thus, the tube 14 has a two-layer structure with the inner tube 24 and the coating layer 35 at the distal end portion, and a three-layer structure with the inner tube 24, the outer tube 26, and the coating layer 35 at the proximal end portion. However, the tube 14 is not limited to a multi-layer structure of two or more layers, but may be a single-layer structure. It would also be acceptable to cover the surfaces of the inner tube 24 and the outer tube 26 with a resin tube. Although the coating layer 35 is much thinner than the inner tube 24 and the outer tube 26, the thickness is exaggerated in the drawings for illustrative purposes.

The perforation head part 12 is attached to the distal end of the tube 14 by the connecting portion 18 being inserted into the distal opening of the tube 14 and being fixed to the tube 14 by means of adhesion or the like. The distal end surface of the tube 14 is abutted against and adhered to the proximal end surface of the perforation head part 12. The perforation head part 12 is arranged so as to cover the distal opening of the lumen 22 of the tube 14, but the lumen 22 is open to the distal side through the through hole 16 that passes through the perforation head part 12 and the connecting portion 18 without being blocked by the perforation head part 12. In the drawings, since the coating layer 35 is shown thicker than it actually is, there is a step between the outer circumferential surface of the perforation head part 12 and the outer circumferential surface of the tube 14, including the coating layer 35. However, actually, the coating layer 35 is extremely thin, so that there are almost no steps between the outer circumferential surface of the perforation head part 12 and the outer circumferential surface of the tube 14. Besides, the outer diameter dimension of the distal end portion of the tube 14, including the coating layer 35, and the outer diameter dimension of the perforation head part 12 can be set to be the same. To give a specific example of such an embodiment, the step between the outer circumferential surfaces of the tube 14 including the coating layer 35 and the perforation head part 12 at their connected part can be substantially eliminated by forming the distal end portion of the coating layer 35 to become thinner by gradually decreasing the outer diameter toward the distal end side, for example by heating with a laser, or the like. Alternatively, a tube 14 with an outer diameter that is smaller than the outer diameter of the proximal end of the perforation head part 12 by an amount equivalent to the thickness of the coating layer 35 may be adopted. The substantial absence of such a step between the outer circumferential surfaces can be confirmed, for example, by the adhesion of tissue or the like to the relevant area after the procedure, and the adhesion of foreign matter to the relevant area also means the presence of resistance to the body tissue.

By the perforation head part 12 being attached to the tube 14, the inner tube 24 of the tube 14 is electrically connected to the perforation head part 12. Accordingly, high-frequency energy can be supplied from a high frequency generator 46 (described later) to the perforation head part 12 through the inner tube 24. Although wiring for energization can be provided through the lumen 22, simplification of the structure, reduction in the number of parts and the like can be achieved by utilizing the inner tube 24 as the wiring for energization.

The perforation head part 12 is exposed at the distal end side with respect to the tube 14, and the connecting portion 18 inserted into the tube 14 is covered by the coating layer 35 of the tube 14. With this configuration, the high-frequency energy supplied through the inner tube 24 is discharged to the outside at the perforation head part 12, while at the connecting portion 18, the discharge of the energy to the outside is prevented by the coating layer 35. This allows the high-frequency energy supplied to the perforation head part 12 to be used efficiently for the perforation of the fossa ovalis A, as described later.

The perforation head part 12 and the connecting portion 18 are arranged on the distal end side with respect to the side holes 32 of the tube 14, and the side holes 32 open on the proximal end side with respect to the perforation head part 12 and the connecting portion 18 without being covered by the perforation head part 12 and the connecting portion 18. In the present practical embodiment, the proximal end of the connecting portion 18 and the distal end of the opening of the side hole 32 are located at approximately the same position in the length direction. However, for example, the side hole 32 may be located proximally away from the proximal end of the connecting portion 18. In the present practical embodiment, the minimum inner diameter dimension of the through hole 16 passing through the perforation head part 12 and the connecting portion 18, and the inner diameter dimension of the side hole 32 of the tube 14 are approximately the same. It is desirable that the inner diameter dimension of the side hole 32 be equal to or greater than the minimum inner diameter dimension of the through hole 16. For example, a side hole 32 having a circular shape of 0.3 mm, or an oval shape of 0.3 mm (minor axis)×0.4 mm (major axis) can be employed with respect to a through hole 16 of 0.1 to 0.3 mm. By setting the inner diameter dimension of side hole 32 to be equal to or greater than the minimum inner diameter dimension of the through hole 16, liquid flow through the side hole 32 is effectively obtained. In addition, the small diameter of the through hole 16 improves the visibility of the perforation head part 12 during contrast imaging. The small diameter of the through hole 16 also makes it easier to obtain a large area at the distal end of the perforation head part 12, thereby enabling a large area of the body tissue to be cauterized by the perforation head part 12.

The distal end of the opening of the side hole 32 is located within the range of 0.5 to 5.0 mm from the distal end of the perforation head part 12 to the proximal end side, more suitably within the range of 1 to 4 mm. In the present practical embodiment, the proximal end of the connecting portion 18 and the distal end of the opening of the side hole 32 are located at approximately the same position in the length direction. Thus, the distance from the distal end of the perforation head part 12 to the distal end of the opening of the side hole 32 is approximately equal to the sum of the length L1 of the perforation head part 12 and the length L2 of the connecting portion 18.

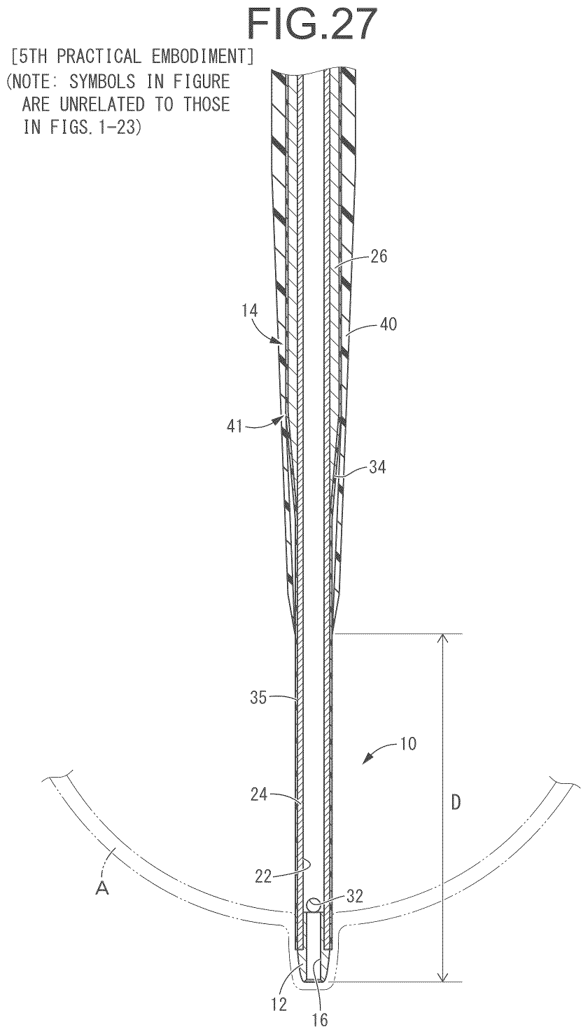

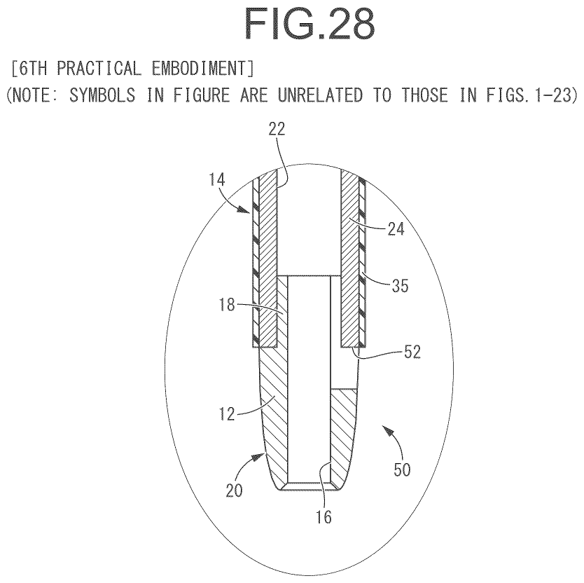

The high-frequency needle 10 with such a structure is used, for example, in a procedure to form a patent foramen in the fossa ovalis A (see FIG. 27) of the atrial septum (the Brockenbrough method). The high-frequency needle 10 is inserted into a sheath 36 as shown in FIG. 26, for example, and the tube 14 is connected to a transducer 38 on the proximal side. In the sheath 36, an instrument other than the high-frequency needle 10, such as a dilator 40 (see FIG. 27), may be inserted if necessary. In the present practical embodiment, the dilator 40 arranged in the sheath 36 is externally combined with the high-frequency needle 10. In FIG. 26, for illustrative purposes, the high-frequency needle 10 is shown inserted through the dilator 40, which has been inserted into the sheath 36 (dilator in sheath), outside the body. Indeed, the high-frequency needle 10 is to be inserted through the dilator 40, which has been arranged in the sheath 36, after the sheath 36 is inserted into the body by a guide wire (not shown).

The dilator 40 is made of resin, such as polyethylene, and has an approximately cylindrical shape, as shown in FIG. 27, with the outer circumferential surface of at least the distal portion gradually increasing in diameter from the distal end to the proximal end. The dilator 40 has a function for facilitating the insertion of the therapeutic catheter by being inserted into the patent foramen formed in the fossa ovalis A by the high-frequency needle 10 thereby pushing open the patent foramen. Regarding the dilator 40 of the present practical embodiment, the inner circumferential surface of the distal side portion has a tapered shape increasing in diameter from the distal end toward the proximal end, and the inner diameter dimension at the distal end is smaller than the maximum outer diameter dimension of the outer tube 26 of the high-frequency needle 10. With this configuration, the tube 14 including the outer tube 26 is prevented from slipping out of the dilator 40 toward the distal end side. Accordingly, the contact between the outer tube 26 and the inner circumferential surface of the dilator 40 constitutes a projection length limiting mechanism 41 that limits the projection length of the tube 14 from the dilator 40. The projection length limiting mechanism 41 limits the maximum distance D from the distal end of the dilator 40 to the distal end of the perforation head part 12 when the high-frequency needle 10 projects most from the dilator 40 toward the distal end, suitably within the range of 3 to 20 mm, more suitably 8 to 15 mm. This avoids the trouble of the side holes 32 being covered by the dilator 40 due to insufficient projection length of the high-frequency needle 10 from the dilator 40, and also prevents the high-frequency needle 10 from projecting excessively from the dilator 40 and touching the body tissue other than the fossa ovalis A, the target of the perforation.

The transducer 38 converts the pressure in the lumen 22 of the tube 14 into an electrical signal to display on a monitor 42. A pressurizing bag 44 containing physiological saline is connected to the lumen 22. The pressurizing bag 44 supplies physiological saline to the lumen 22 at a pressure equal to or greater than the blood pressure, thereby preventing blood from flowing through the lumen 22 into the transducer 38. Pressurization of the lumen 22 by the pressurizing bag 44 can be controlled, for example, according to the intracardiac pressure acting in the lumen 22.

A high frequency generator 46 is connected to the tube 14 of the high-frequency needle 10. High-frequency energy generated by the high frequency generator 46 is supplied to the perforation head part 12 through the inner tube 24 of the tube 14, causing the perforation head part 12 to perform perforation operation. A counter electrode plate 48 connected to the high frequency generator 46 is attached to the patient to prevent electric shock to the patient during the high frequency energization.

Since the Brockenbrough method is conventionally known, a detailed description will be omitted. First, the practitioner inserts the sheath 36, into which the dilator 40 is inserted, to the right atrium along a guide wire (not shown) that has been previously inserted through a somatic lumen, such as a blood vessel. Next, the practitioner inserts the high-frequency needle 10 into the right atrium through the dilator 40 inserted into the sheath 36 from which the guidewire has been removed. Regarding the high-frequency needle 10 inserted into the sheath 36, the distal opening of the lumen 22 opens into the right atrium through the through hole 16 of the perforation head part 12, and the side holes 32 of the lumen 22 open into the right atrium. Thus, the intracardiac pressure in the right atrium is measured by the transducer 38 via the lumen 22 and displayed on the monitor 42.

Subsequently, the practitioner projects the perforation head part 12, which is the distal end of the high-frequency needle 10, from the distal end of the dilator 40 and presses it against the fossa ovalis A of the atrial septum (see FIG. 27). The distal opening of the through hole 16 of the perforation head part 12 is blocked by the press against the fossa ovalis A, and the distal opening of the lumen 22 is blocked. When the communication area of the lumen 22 with the right atrium is reduced by the blockage of the distal opening, the pressure in the lumen 22, which is controlled by the pressurizing bag 44 according to the intracardiac pressure acting in the lumen 22, decreases. By so doing, the detection of the decrease in the internal pressure of the lumen 22 by the transducer 38 is displayed on the monitor 42, and the press of the perforation head part 12 against the fossa ovalis A can be grasped by the change in pressure.

The lumen 22 communicates with the right atrium through the side holes 32 even when the distal opening of the through hole 16 is blocked. The side holes 32 are located to the distal side with respect to the distal end of the dilator 40 and open without being covered by the dilator 40 when the high-frequency needle 10 projects from the dilator 40 to the distal end side and is pressed against the fossa ovalis A. Besides, when the perforation head part 12 is pressed against the fossa ovalis A, the fossa ovalis A deforms in a flexural manner so as to wrap around the distal end portion of the high-frequency needle 10. However, the side holes 32 formed on the proximal end side with respect to the perforation head part 12 are kept open without being covered by the fossa ovalis A (see FIG. 27). In particular, the distal end of the opening of the side hole 32 is positioned at least 0.5 mm away from the distal end of the perforation head part 12 to the proximal end side. This configuration prevents the side hole 32 from being blocked by the fossa ovalis A which has deformed in a flexural manner. In this way, by providing the side hole 32 at the position which is not covered by the fossa ovalis A, the lumen 22 of the tube 14 is kept in a communicating state with the outside (the interior of the atrium) and the function of releasing a fluid such as physiological saline through the lumen 22 into the atrium will be stably maintained.

By supplying high-frequency electric power to the perforation head part 12, which is pressed against the fossa ovalis A, the perforation head part 12 performs perforation operation and forms a patent foramen in the fossa ovalis A. Here, the projection length limiting mechanism 41 limits the projection length D of the high-frequency needle 10 from the dilator 40 to the distal end. Thus, for example, even if the high-frequency needle 10 projecting from the dilator 40 passes through the patent foramen of the fossa ovalis A, the perforation head part 12 of the high-frequency needle 10 is never accidentally strongly pressed against the wall of the left atrium.

When the perforation head part 12 passes through the fossa ovalis A, the blockage of the through hole 16 of the perforation head part 12 by the fossa ovalis A is released, and the intracardiac pressure acts on the lumen 22 not only through the side holes 32 but also through the through hole 16. The distal end portion of the high-frequency needle 10, which has passed through the patent foramen of the fossa ovalis A, is inserted into the left atrium. Thus, the intracardiac pressure of the left atrium is exerted on the lumen 22 through the through hole 16 and the side holes 32, and in association therewith, the lumen 22 is pressurized by the pressurizing bag 44, causing the pressure in the lumen 22 to increase according to the intracardiac pressure of the left atrium. Then, the pressure in the left atrium is detected by transducer 38 and displayed on the monitor 42.

Therefore, during the perforation of the fossa ovalis A by the high-frequency needle 10, due to the decrease in the internal pressure of the lumen 22 detected by the transducer 38, it is possible to grasp that the perforation head part 12 of the high-frequency needle 10 is pressed against the fossa ovalis A, and to start the perforation operation by supplying high-frequency energy. When the internal pressure of the lumen 22 detected by the transducer 38 increases after the above-mentioned decrease and the increased internal pressure indicates the internal pressure of the left atrium, it is possible to grasp that the perforation of the fossa ovalis A by the high-frequency needle 10 is completed and that the distal end of the high-frequency needle 10 has been inserted into the left atrium.

In this way, in the high-frequency needle 10 according to the present practical embodiment, the through hole 16, which is the distal opening of the lumen 22, is switched between communication and blockage according to the state of perforation. Therefore, by measuring the pressure in the lumen 22 that changes due to communication and blockage of the through hole 16, it is possible to accurately grasp the state before perforation where the distal end of the high-frequency needle 10 is inserted into the right atrium, the state during perforation where the distal end of the high-frequency needle 10 is pressed against the fossa ovalis A, and the state after perforation where the distal end of the high-frequency needle 10 is inserted into the left atrium.

Moreover, even when the through hole 16 is blocked by the fossa ovalis A, the side holes 32 are kept in a communicating state, so that the function of administering liquids (for example, contrast media, drugs, etc.) into the atrium through the lumen 22 is stably maintained by the side holes 32. For example, it would also be possible to suction fluid in the atrium into the lumen 22 through the through hole 16 and/or the side holes 32.

The position of the perforation head part 12 can be confirmed not only by pressure changes in the lumen 22, but also by observation with X-ray transmission images or ultrasonic images. In particular, the state of perforation can also be confirmed by observing the release of fluid (such as a contrast medium) from the lumen 22 through the through hole 16 and/or the side holes 32.

While the present invention has been described in detail hereinabove in terms of the practical embodiments, the invention is not limited by the specific description thereof. For example, the preceding practical embodiment describes an example in which the body tissue perforation device is used for perforation of the atrial septum (the fossa ovalis), but the body tissue for which a patent foramen is formed is not limited to the atrial septum.

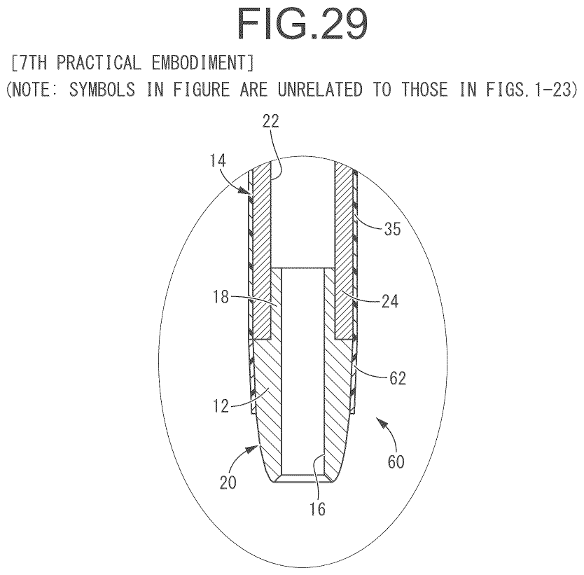

### [Sixth Practical Embodiment (FIG. 28)] (Note: The Symbols in the Figures are Unrelated to Those in FIGS. 1-23)

Whereas the preceding practical embodiment shows an example in which the side hole 32 opens onto the outer circumferential surface of the tube 14, the side hole may be located, for example, on the distal side with respect to the distal end of the tube 14. Specifically, the side hole may open onto the outer circumferential surface between the distal end of the tube 14 and the perforation head part 12, such as a side hole 52 provided to a high-frequency needle 50 serving as a body tissue perforation device shown in FIG. 28. Besides, the side hole may also be formed through the peripheral wall of the through hole 16, for example, so as to open onto the outer circumferential surface of the perforation head part 12.

The side hole can also be formed at multiple locations in the length direction. For example, the side hole may be formed at multiple locations in the length direction at the distal end portion of inner tube 24, or the side hole may be formed at each of the distal end portion of inner tube 24 and the perforation head part 12. Also, the side hole may be formed between the distal end of the inner tube 24 and the perforation head part 12, as well as at the inner tube 24 and/or the perforation head part 12.

### [Seventh Practical Embodiment (FIG. 29)] (Note: The Symbols in the Figures are Unrelated to Those in FIGS. 1-23)

Further, an insulating layer 62 covering the outer circumferential surface on the proximal end side of the perforation head part 12 can also be provided, as in a high-frequency needle 60 serving as a body tissue perforation device shown in FIG. 29. The insulating layer 62 can be provided, for example, by an electrically insulating tube fitted onto the outer circumferential surface on the proximal end side of the perforation head part 12, or by an electrically insulating film formed so as to cover the outer circumferential surface on the proximal end side of the perforation head part 12 by means of coating, vapor deposition, or the like. By providing such an insulating layer 62 covering the outer circumferential surface of the proximal end side of the perforation head part 12, the high-frequency energy supplied to the perforation head part 12 can be concentrated on the distal end portion of the perforation head part 12 to improve energy efficiency when perforating the body tissue (such as the fossa ovalis A). The insulating layer 62 can be formed integrally with the coating layer 35 of the tube 14, for example. In the high-frequency needles 50, 60 shown in FIGS. 28 and 29, components and parts that are substantially identical with those in the first practical embodiment will be assigned like symbols and not described in any detail.

It would be acceptable as long as the perforation head part emits energy for perforation, and the perforation head part is not necessarily limited to the one operated by high-frequency energy. For example, the perforation head part can also be constituted by a temperature-raising body (by thermal energy) that cauterizes a body tissue by heating it with an electric current or laser, etc., and a combination of multiple energies can also be employed. In addition to the mode in which energy is supplied from outside the device, the device may also be equipped with means for storing energy inside. Furthermore, the perforation head part is not particularly limited in terms of material, hardness, shape, etc., and may be made of the same material as the inner tube, for example. The perforation head part is not limited to the mode of the preceding practical embodiment in which the perforation head part is fixed to the tube 14 with the connecting portion 18 inserted in the distal end portion of the tube 14. For example, the perforation head part may be fixed to the tube 14 with the connecting portion externally fitted onto the distal end portion of the tube 14, or the connecting portion may be omitted and the perforation head part may be fixed to the tube 14 by its proximal end surface being abutted against and fastened to the distal end surface of the tube 14.

The preceding practical embodiment illustrates the perforation head part 12 that functions as a radiopaque marker corresponding to an X-ray imaging device. However, instead of or in addition to the function as a radiopaque marker, the perforation head part may have a function as, for example, an echo marker corresponding to an ultrasonic imaging device or a magnetic marker corresponding to a magnetic resonance imaging device.

The projection length limiting mechanism for limiting the projection length of the tube 14 from the dilator 40 may be constituted by, for example, a step provided on the inner circumferential surface of the dilator 40 and a step provided on the outer circumferential surface of the tube 14 being caught by each other (by mechanical engagement). Besides, the outer tube 26 is not essential, and for example, the projection length limiting mechanism as described above can also be constituted by integrally forming a protrusion on the outer circumferential surface of the inner tube 24 or by externally attaching a ring fitting onto the inner tube 24 so as to provide a projecting portion that catches on the inner circumferential surface of the dilator 40. Moreover, it is not necessarily essential for the projection length limiting mechanism to be provided inside the dilator 40. For example, the projection length of the tube 14 from the dilator 40 may be limited by a projecting portion that projects from the outer circumferential surface of the tube 14 being engaged with the proximal end surface of the dilator 40.

### KEYS TO SYMBOLS

### First to Fourth Practical Embodiments (FIGS. 1-23)

10 high-frequency needle (perforation device, first practical embodiment)
12 shaft
14 perforation head part
16 operation handle
18 through hole
20 connecting portion
22 distal end tip
24 lumen
26 side hole
28 shaft curved part
30 first positioning protrusion (positioning part)
32 coating layer
34 marker part
35 rib-shaped part
36 internal flow path
38 distal end connecting part
40 proximal end port part
42 wiring
44 curve indicator projection
46 proximal end concave surface
48 distal end inclined surface
49 gripping surface
50 rotational operation assisting surface
52 sheath
54 sheath curved part
56 sheath hub
58 side port
60 second positioning recess
62 dilator
64 dilator hub
66 second positioning protrusion
68 first positioning recess
70 dilator curved part
72 transducer
74 monitor
76 pressurizing bag
78 high frequency generator
80 counter electrode plate
82 perforation device assembly
90 high-frequency needle (perforation device, second practical embodiment)
92 operation handle
100 high-frequency needle (perforation device, third practical embodiment)
102 operation handle
104 rotational operation assisting surface
110 perforation device assembly (another practical embodiment (fourth practical embodiment))
112 high-frequency needle (perforation device)
114 operation handle
116 distal end connecting part

### Fifth to Seventh Practical Embodiments (FIGS. 24-29)

10 high-frequency needle (body tissue perforation device, fifth practical embodiment)
12 perforation head part
14 tube
16 through hole
18 connecting portion
20 distal end tip
22 lumen
24 inner tube
26 outer tube
32 side hole
34 tapered surface
35 coating layer
36 sheath
38 transducer
40 dilator
41 projection length limiting mechanism
42 monitor
44 pressurizing bag
46 high frequency generator
48 counter electrode plate
50 high-frequency needle (body tissue perforation device, sixth practical embodiment)
52 side hole
60 high-frequency needle (body tissue perforation device, seventh practical embodiment)
62 insulating layer

1. An atrial septum perforation device comprising:
a shaft;
a perforation head part that is provided to a distal end of the shaft and is configured to perform perforation operation on a body tissue; and
an operation handle that is provided to a proximal end of the shaft and is configured to be gripped by a practitioner, wherein
a distal end portion of the shaft comprises a shaft curved part that is curved in one direction,
the operation handle includes a rotational operation assisting surface that extends vertically or horizontally when a direction of curving of the shaft curved part is set to a substantially 4 o'clock direction around a body axis parallel line of a patient such that the perforation head part is turned to a fossa ovalis.

2. The atrial septum perforation device according to claim 1, wherein
the operation handle has a plate shape, and
the rotational operation assisting surface is provided on an outer circumferential surface of the operation handle and is inclined relative to a plate thickness direction of the operation handle.

3. The atrial septum perforation device according to claim 2, wherein
on the outer circumferential surface of the operation handle, the rotational operation assisting surface is partially provided in the plate thickness direction of the operation handle, and
on the outer circumferential surface of the operation handle, a gripping surface is provided, the gripping surface being adjacent to the rotational operation assisting surface in the plate thickness direction of the operation handle while extending substantially parallel to the plate thickness direction of the operation handle.

4. The atrial septum perforation device according to claim 2, wherein the plate thickness direction of the operation handle is substantially orthogonal to a plane including a central axis of the shaft curved part.

5. The atrial septum perforation device according to claim 4, wherein
the rotational operation assisting surface is provided on the outer circumferential surface of the operation handle located on a side opposite to the direction of curving of the shaft curved part when viewed in an axial direction from a proximal side to a distal side.

6. The atrial septum perforation device according to claim 1, wherein
the shaft is configured to be inserted through a dilator, and
a marker part is provided on a proximal end side of the shaft, the marker part indicating in a visually confirmable manner that a distal end of the perforation head part is housed in the dilator without protruding from the dilator.

7. The atrial septum perforation device according to claim 1, wherein
the shaft is configured to be inserted through a dilator, and
the shaft includes a positioning part configured to position the shaft with respect to the dilator in a circumferential direction.

8. A body tissue perforation device comprising:
a shaft;
a perforation head part that is provided to a distal end of the shaft and is configured to perform perforation operation on a body tissue; and
an operation handle that is provided to a proximal end of the shaft and is configured to be gripped by a practitioner, wherein
the shaft is configured to be inserted through a dilator, and
a marker part is provided on a proximal end side of the shaft, the marker part indicating in a visually confirmable manner that a distal end of the perforation head part is housed in the dilator without protruding from the dilator.

9. The body tissue perforation device according to claim 8, wherein alignment between the marker part and a proximal end of the dilator indicates that the distal end of the perforation head part is housed in the dilator.

10. The body tissue perforation device according to claim 8, wherein
a surface of the shaft is covered by an electrically insulating coating material, and
the electrically insulating coating material is partially different in color from other portions such that the marker part is provided on the surface of the shaft.

11. The body tissue perforation device according to claim 8, further comprising an extended part extending out on an outer circumferential surface of the shaft from the operation handle, wherein
the extended part is allowed to be inserted in a proximal end part of the dilator, and
the extended part constitutes the marker part.

12. The body tissue perforation device according to claim 8, wherein the marker part visibly indicates that the distal end of the perforation head part is housed in the dilator without protruding from the dilator before a retraction amount of the distal end of the perforation head part into the dilator exceeds 20 mm.

13. A perforation device assembly comprising:
a body tissue perforation device comprising:
a shaft;
a perforation head part that is provided to a distal end of the shaft and is configured to perform perforation operation on a body tissue; and
an operation handle that is provided to a proximal end of the shaft and is configured to be gripped by a practitioner;
a dilator through which the shaft of the body tissue perforation device is inserted;
a sheath through which the dilator is inserted;
a first circumferential positioning mechanism mutually positioning the shaft of the body tissue perforation device and the dilator in a circumferential direction; and
a second circumferential positioning mechanism mutually positioning the dilator and the sheath in the circumferential direction.

14. The perforation device assembly according to claim 13, wherein
the shaft of the body tissue perforation device includes a shaft curved part,
the dilator includes a dilator curved part that has a curved shape corresponding to that of the shaft curved part and through which the shaft curved part is inserted, and
the sheath includes a sheath curved part that has a curved shape corresponding to that of the dilator curved part and through which the dilator curved part is inserted.

15. (canceled)

16. (canceled)

17. (canceled)

18. (canceled)

19. (canceled)

20. (canceled)

21. (canceled)

22. (canceled)

23. (canceled)
